(19) United States
(12) Patent Application Publication
(10) Pub. No.: US 2026/0102137 A1
(43) Pub. Date: Apr. 16, 2026

(54) FORMULA FOR DETERMINING WATER VAPOR THERMAL THERAPY INJECTIONS

(71) Applicant: Javier Castillo-Ortiz, Ponce, PR (US)

(72) Inventor: Javier Castillo-Ortiz, Ponce, PR (US)

(21) Appl. No.: 19/346,543

(22) Filed: Sep. 30, 2025

Related U.S. Application Data
(60) Provisional application No. 63/707,797, filed on Oct. 16, 2024.

(51) Int. Cl. A61B 8/08

(52) U.S. Cl. CPC A61B 8/08; A61B 2576/02

(57) ABSTRACT

Method for determining a number of water vapor thermal therapy (WVTT) injections for treatment of benign prostate hyperplasia. It comprises obtaining a prostate volume of a patient and calculating a R.I.D. Score by multiplying the prostate volume by a R.I.D. Score factor, wherein the R.I.D. Score indicates the number of WVTT injections required for treatment. The R.I.D. Score factor may be within a range of 0.0025 to 0.6 injections per cubic centimeter, preferably a range of 0.025 to 0.1 injections per cubic centimeter, and more preferably a range of 0.045 to 0.055 injections per cubic centimeter. Obtaining the prostate volume may comprise using at least one imaging technique selected from Computed Tomography Scan, Magnetic Resonance Imaging, and Ultrasound. The method may further comprise adjusting the R.I.D. Score to account for prostate anatomical variations of the patient. A system and non-transitory computer-readable medium for implementing the method are also disclosed.

### CROSS-REFERENCE TO RELATED APPLICATIONS

This application claims the benefit of U.S. Provisional Ser. No. 63/707,797, filed on Oct. 16, 2024, which is incorporated by reference herein in its entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates in general to the field of medical treatment planning systems for benign prostatic hyperplasia, and more particularly to a method and system for determining the number of water vapor thermal therapy injections required for treatment using a calculated injection density score based on prostate volume.

### 2) Description of Related Art

Benign prostatic hyperplasia (BPH) is a common condition affecting men as they age, involving non-cancerous enlargement of the prostate gland that can lead to urinary symptoms and decreased quality of life. Various treatment options exist for BPH, ranging from medication to surgical interventions, with treatment selection typically based on factors such as prostate size, symptom severity, and patient characteristics.

Minimally invasive treatments have gained attention in recent years as alternatives to more invasive surgical options. Water vapor thermal therapy (WVTT) represents one such approach, involving the delivery of controlled doses of water vapor directly into prostate tissue to reduce its size and alleviate symptoms. This procedure offers potential advantages over more invasive surgical options in terms of reduced complications and faster recovery times.

Treatment planning for WVTT procedures traditionally relies on methods performed during the procedure itself, such as measuring prostatic urethral length through rigid cystoscopy. During these procedures, healthcare providers typically determine the number of water vapor injections based on anatomical measurements taken in real-time. This approach can lead to patient discomfort, increased procedural complexity, and uncertainty for both healthcare providers and patients regarding the extent of treatment needed.

Current methods for determining injection parameters may contribute to procedural discomfort and post-treatment complications. The insertion of rigid cystoscopes and real-time measurement procedures can cause patient discomfort and may increase the risk of complications such as urinary retention, irritative symptoms, and other adverse events. Additionally, the inability to predict treatment parameters in advance can create anxiety for patients and limit the ability of healthcare providers to optimize treatment planning.

There exists a general need for improved methods of treatment planning for WVTT procedures that can enhance patient comfort, reduce procedural risks, and provide greater predictability of outcomes.

As the field of minimally invasive BPH treatments continues to evolve, developing more refined and patient-centric approaches to treatment planning and delivery remains a goal for improving outcomes and expanding access to effective therapies.

These and other objects, features, and advantages of the present invention will become more readily apparent from the attached drawings and the detailed description of the preferred embodiments, which follow.

### SUMMARY OF THE INVENTION

Bearing in mind the problems and deficiencies of the prior art, it is therefore an object of the present invention to provide a method for determining a number of water vapor thermal therapy (WVTT) injections for treatment of benign prostate hyperplasia.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for determining a number of water vapor thermal therapy (WVTT) injections for treatment of benign prostate hyperplasia is provided. The method comprises obtaining a prostate volume of a patient. The method further comprises calculating a R.I.D. Score by multiplying the prostate volume by a R.I.D. Score factor, wherein the R.I.D. Score indicates the number of WVTT injections required for treatment.

According to another aspect of the present disclosure, a system for determining a number of water vapor thermal therapy (WVTT) injections for treatment of benign prostate hyperplasia is provided. The system includes an imaging device configured to obtain a prostate volume of a patient. The system further includes a processor configured to calculate a R.I.D. Score by multiplying the prostate volume by a R.I.D. Score factor, wherein the R.I.D. Score indicates the number of WVTT injections required for treatment.

According to another aspect of the present disclosure, a non-transitory computer-readable medium storing instructions that, when executed by a processor, cause the processor to perform a method for determining a number of water vapor thermal therapy (WVTT) injections for treatment of benign prostate hyperplasia is provided. The method includes receiving a prostate volume of a patient. The method further includes calculating a R.I.D. Score by multiplying the prostate volume by a R.I.D. Score factor, wherein the R.I.D. Score indicates the number of WVTT injections required for treatment.

The foregoing general description of the illustrative embodiments and the following detailed description thereof are merely exemplary aspects of the teachings of this disclosure and are not restrictive.

The above and other objects, which will be apparent to those skilled in the art, are achieved in the present invention which is directed to a method for determining a number of water vapor thermal therapy (WVTT) injections for treatment of benign prostate hyperplasia, comprising:
a. obtaining a prostate volume of a patient; and
b. calculating a R.I.D. Score by multiplying the prostate volume by a R.I.D. Score factor, wherein the R.I.D. Score indicates the number of WVTT injections required for treatment.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a flowchart of a surgical therapy process for prostate treatment, according to aspects of the present disclosure.

FIG. 2 shows a cross-sectional view of a prostate with a delivery device positioned within the urethra, according to aspects of the present disclosure.

FIG. 3 illustrates a diagram showing anatomical structure and measurements for water vapor thermal therapy treatment, according to aspects of the present disclosure.

FIG. 4 depicts a medical scan showing measurement of a prostatic urethral angle, according to aspects of the present disclosure.

FIG. 5A depicts a scan showing multiple injection sites along the prostatic urethra of FIG. 3, according to aspects of the present disclosure.

FIGS. 5B-5D show magnetic resonance imaging scans of injection sites in the prostate of FIG. 2, according to aspects of the present disclosure.

FIG. 6 shows a flowchart illustrating a cohort study process with sequential steps, according to aspects of the present disclosure.

FIG. 7A depicts a symptom scoresheet for evaluating urinary symptoms, according to aspects of the present disclosure.

FIG. 7B depicts a pain score numerical rating scale, according to aspects of the present disclosure.

FIG. 8 depicts a graph showing RID Score versus AUA SS Change at four weeks, according to aspects of the present disclosure.

FIG. 9 depicts a graph showing RID Score versus AUA SS Change at four months, according to aspects of the present disclosure.

FIG. 10 depicts a graph showing AUA symptom score versus time after TWVT, according to aspects of the present disclosure.

FIG. 11 depicts a graph showing AUA symptom score with baseline measurement, according to aspects of the present disclosure.

FIG. 12 depicts a graph showing AUA symptom score changes at four weeks, according to aspects of the present disclosure.

FIG. 13 depicts a graph showing AUA symptom score changes at four months, according to aspects of the present disclosure.

FIG. 14 depicts a pain score rating chart with numerical scale, according to aspects of the present disclosure.

FIG. 15 depicts a pain score rating chart showing categories of discomfort, according to aspects of the present disclosure.

FIG. 16 depicts a graph showing relationship between prostatic urethral angles and pain ratings, according to aspects of the present disclosure.

FIG. 17 depicts a chart showing procedure discomfort values versus prostatic urethral angle, according to aspects of the present disclosure.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

Prior to a discussion of the preferred embodiment of the invention, it should be understood that while the features and advantages of the invention are illustrated in terms of a method for determining a number of water vapor thermal therapy (WVTT) injections for treatment of benign prostate hyperplasia

The following description sets forth exemplary aspects of the present disclosure. It should be recognized, however, that such description is not intended as a limitation on the scope of the present disclosure. Rather, the description also encompasses combinations and modifications to those exemplary aspects described herein.

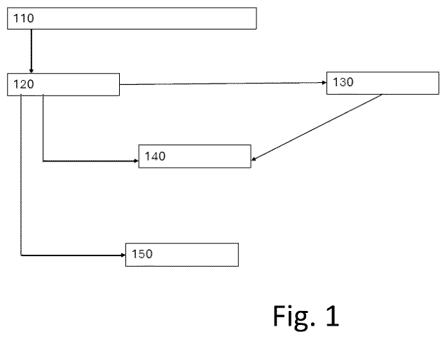

Referring to FIG. 1, a method 110 for determining water vapor thermal therapy (WVTT) injections begins with surgical therapy considerations for benign prostatic hyperplasia treatment. The method 110 proceeds through a step 120 where assessment of prostate size occurs via imaging or cystoscopy. Based on prostate size determinations, the process follows different pathways including a step 130 for large prostates involving simple prostatectomy or laser enucleation procedures, a step 140 for average-sized prostates incorporating various minimally invasive techniques including WVTT, and a step 150 for small prostates utilizing transurethral procedures. The R.I.D. Score formula system provides a computational approach for predicting the number of WVTT injections based on prostate volume measurements and calculated density factors, eliminating traditional measurement protocols that require invasive bladder entry and manual increment measurements during procedures.

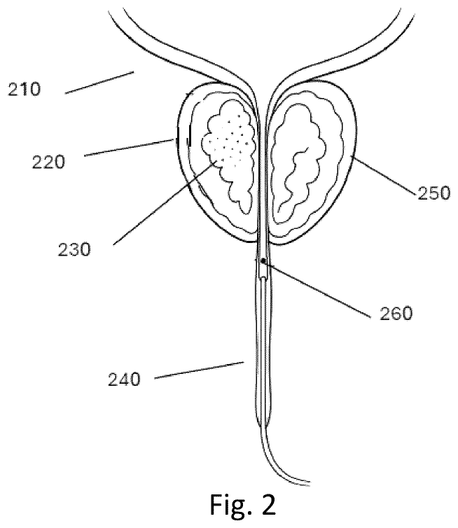

With reference to FIG. 2, the anatomical context for WVTT procedures involves a bladder 210 positioned above a prostate 250 containing prostate tissue 230. Steam 220 represents the water vapor delivered through a urethra 240 via a delivery device 260 during treatment. The R.I.D. Score calculation system accounts for these anatomical relationships by incorporating prostate volume measurements obtained through non-invasive imaging techniques. Water vapor delivery occurs at approximately 103 degrees Celsius, with each WVTT injection delivering approximately 0.5 mL of water vapor over 9 seconds. The R.I.D. Score factor ranges from 0.0025 to 0.6 injections per cubic centimeter, with preferred ranges of 0.025 to 0.1 injections per cubic centimeter and more preferred ranges of 0.045 to 0.055 injections per cubic centimeter.

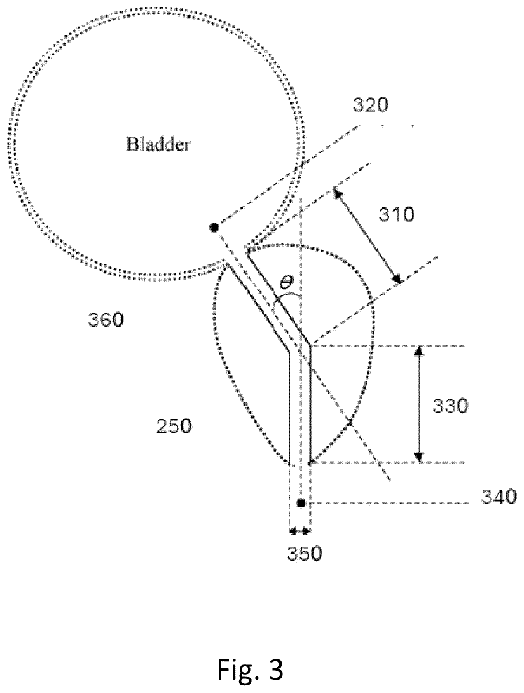

As shown in FIG. 3, anatomical measurements include a prostatic urethra 310 extending from the bladder region to a distal urethra 330, with an initial pressure point 320 and a secondary pressure point 340 marking measurement locations. A urethral diameter 350 and a urethral angle 360 provide geometric parameters for treatment planning. The normal prostatic urethral angle measures 35 degrees, and the prostatic urethral angle measurement serves to predict procedure discomfort levels. The R.I.D. Score system incorporates these anatomical variations through adjustments that account for individual patient prostate characteristics. In some cases, the R.I.D. Score calculation includes a R.I.D.+Score that incorporates additional factors related to prostate anatomical variations beyond basic volume measurements.

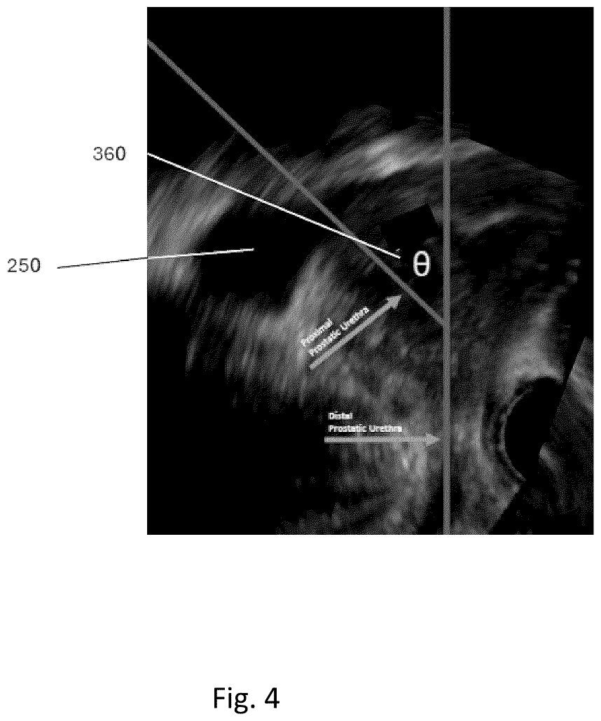

Referring to FIG. 4, the prostate 250 demonstrates the urethral angle 360 measurement used in treatment planning calculations. The R.I.D. Score factor calculation involves dividing the average number of WVTT injections by the average prostate volume, providing a standardized approach to treatment prediction. Testing has established that a R.I.D. Score factor of 0.05 injections per cubic centimeter provides effective treatment outcomes. The system eliminates the need to measure 1 cm increments from the bladder neck during procedures and reduces the need to enter the bladder during WVTT procedures, thereby reducing procedural complexity and patient discomfort.

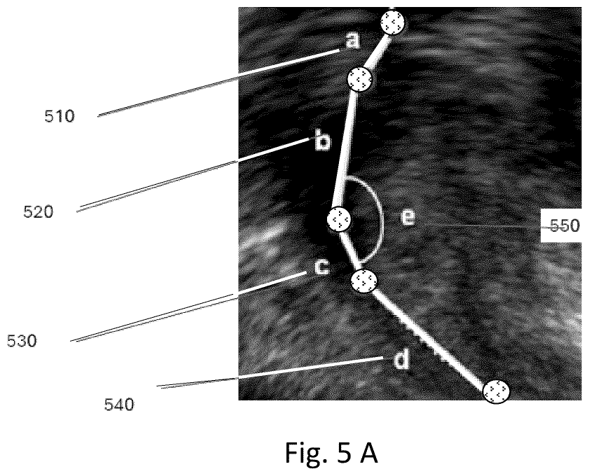

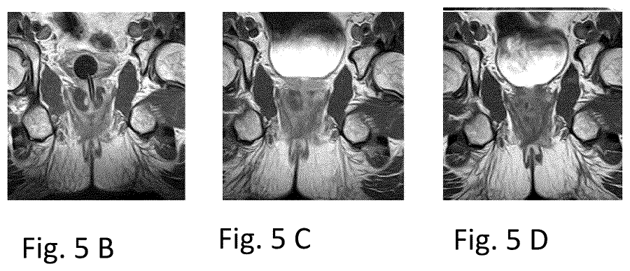

As illustrated in FIG. 5A, injection site planning involves a first injection site 510, a second injection site 520, a third injection site 530, and a fourth injection site 540 positioned along the prostatic urethra. A prostatic angle 550 influences injection placement and treatment effectiveness. The R.I.D. Score system accounts for these spatial relationships through volume-based calculations that predict the total number of injection sites needed for effective treatment. FIGS. 5B-5D show additional imaging perspectives of injection sites that demonstrate the three-dimensional nature of prostate anatomy considered in R.I.D. score calculations.

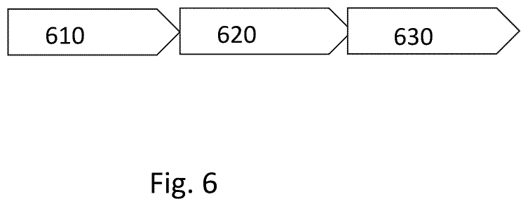

With reference to FIG. 6, clinical validation involves a first step 610 encompassing single-surgeon experience with procedures conducted between October 2023 and June 2024. A second step 620 details patient exclusion criteria, specifically four patients excluded based on non-standard pain protocols and chronic Foley catheter usage. A third step 630 involves measurement and analysis using 7.5 MHz ultrasound with a transrectal probe, along with statistical analysis methods including linear regression models and paired T-tests conducted using SPSS software. The clinical study data demonstrates applicability to prostates with volumes ranging from 18 cc to 93 cc, establishing the broad range of prostate sizes suitable for R.I.D. Score-based treatment planning.

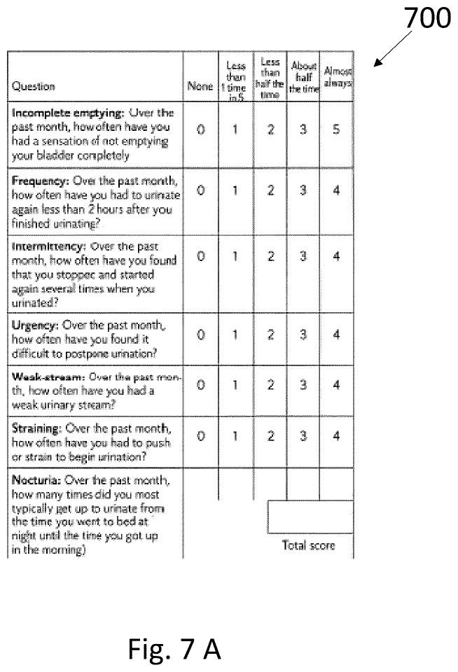

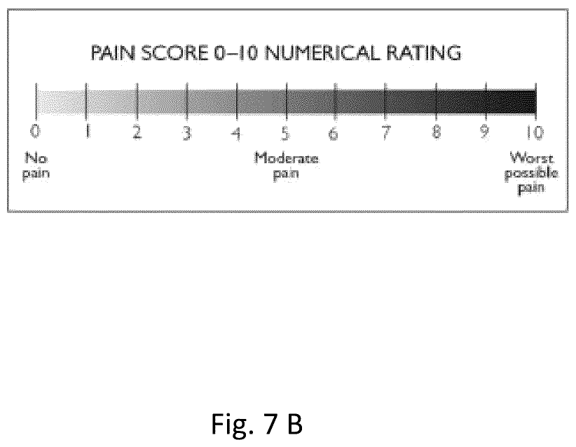

Referring to FIG. 7A, a symptom scoresheet 700 provides standardized assessment tools for evaluating treatment outcomes. The scoresheet 700 contains multiple questions related to urinary function with corresponding numerical rating scales ranging from 0 to 5, allowing patients to indicate symptom frequency over specified time periods. FIG. 7B depicts a pain assessment scale ranging from 0 to 10, with gradations from “No pain” to “Worst possible pain” that correlate with prostatic urethral angle measurements for predicting procedure discomfort levels.

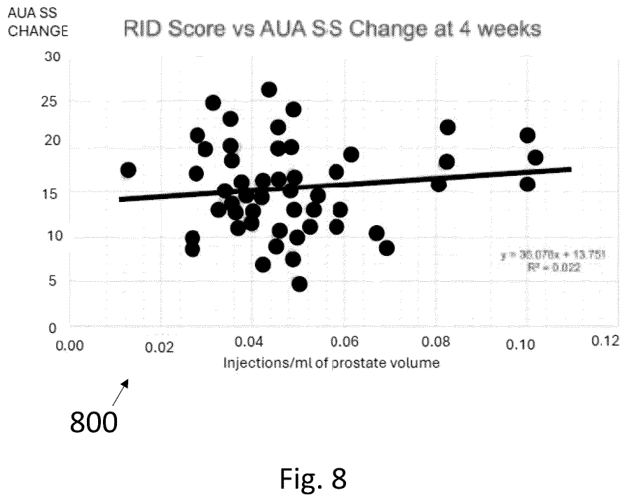

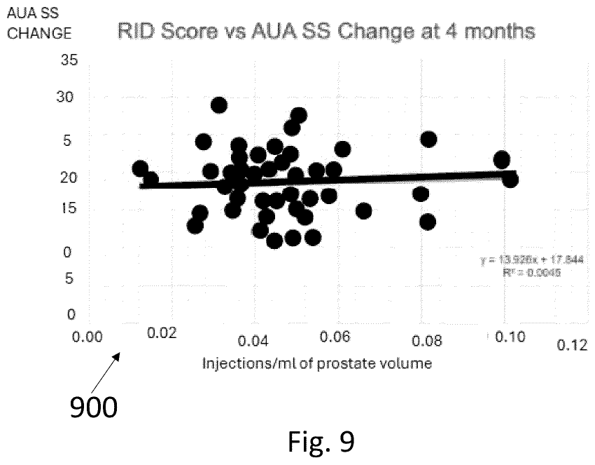

As shown in FIG. 8, statistical analysis demonstrates relationships between R.I.D. Score values and symptom improvement at 4 weeks post-treatment. The scatter plot displays data points with a linear regression line showing the correlation between injections per milliliter of prostate volume and symptom score changes. FIG. 9 presents a graph 900 showing similar relationships at 4 months post-treatment, with the graph 900 displaying scatter plot data and linear regression analysis demonstrating sustained treatment effectiveness over extended time periods.

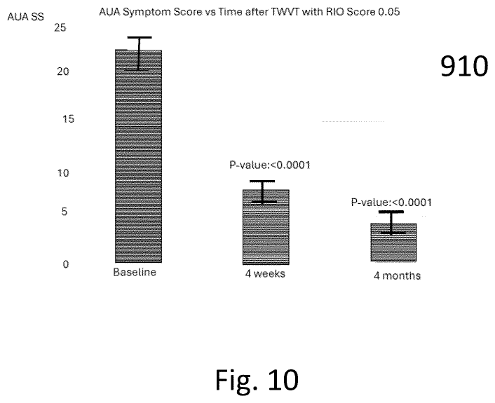

Referring to FIGS. 10, 11, 12, and 13, treatment outcome data demonstrates symptom score improvements over time following WVTT procedures using R.I.D. Score calculations. The data shows baseline symptom scores of approximately 22.56±3.70, with significant reductions to approximately 7 at 4 weeks and further reductions to approximately 4 at 4 months. Statistical significance indicators show P-values of less than 0.0001 for both 4-week and 4-month measurements, establishing the effectiveness of R.I.D. Score-based treatment planning.

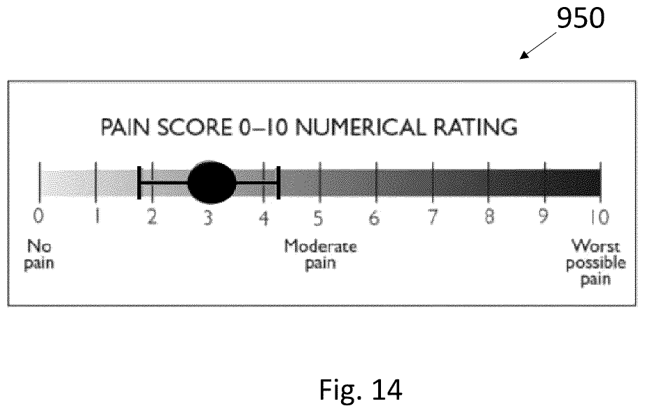

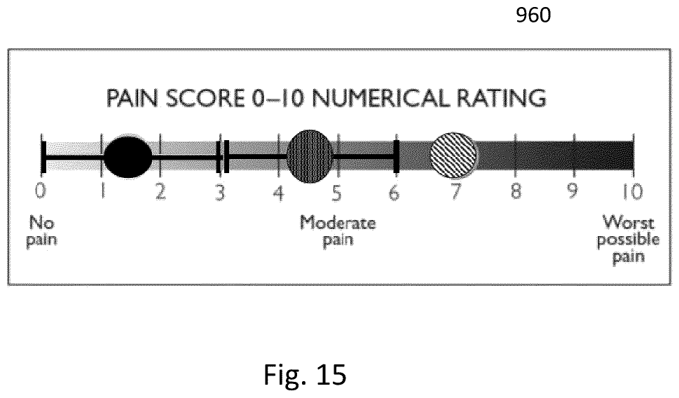

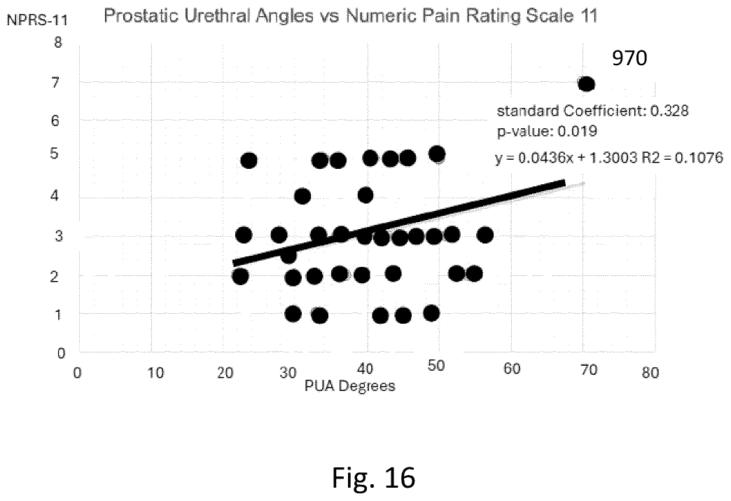

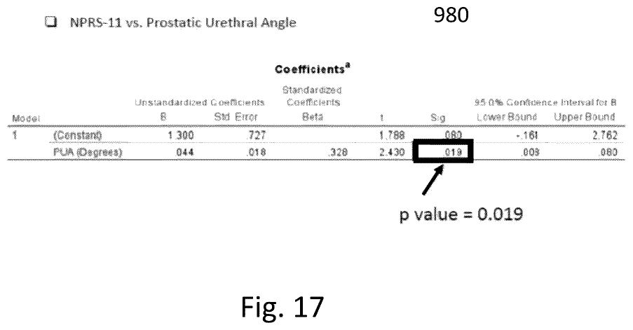

As illustrated in FIGS. 14 and 15, pain assessment charts demonstrate the relationship between prostatic urethral angle measurements and patient discomfort levels during procedures. The charts categorize discomfort levels as mild (0-3), moderate (4-6), and severe (7-10) on numerical rating scales. FIG. 16 shows the correlation between prostatic urethral angles and numeric pain rating scale scores, with statistical analysis indicating a positive correlation described by the linear equation y=0.0436x+1.3003. FIG. 17 presents a procedure discomfort chart 980 showing the relationship between NPRS-11 scores and prostatic urethral angle measurements, including coefficients and statistical data with standardized and unstandardized coefficients, standard error values, and confidence intervals.

The R.I.D. Score system incorporates imaging techniques selected from Computed Tomography Scan, Magnetic Resonance Imaging, and Ultrasound for obtaining prostate volume measurements. Ultrasound imaging utilizes a transrectal probe at 7.5 MHz frequency for accurate volume determination. The system includes an imaging device configured to obtain prostate volume measurements and a processor configured to calculate R.I.D. Score values based on the measured volume and predetermined score factors. The imaging device encompasses at least one of a Computed Tomography scanner, a Magnetic Resonance Imaging device, and an Ultrasound device, providing flexibility in clinical implementation.

Implementation of the R.I.D. Score system involves a non-transitory computer-readable medium storing instructions that, when executed by a processor, cause the processor to perform the method for determining WVTT injections. The computational approach reduces the risk of acute urinary retention from 11-32% observed with standard protocols to 0% through more precise injection number determination. The system provides predictable treatment planning that accounts for individual patient anatomy while maintaining standardized calculation methods across different clinical settings and patient populations.

Referring to FIG. 1, the method 110 establishes a systematic approach for surgical therapy selection based on prostate size assessment and anatomical considerations. The step 120 involves comprehensive evaluation of prostate dimensions through imaging modalities or direct cystoscopic examination to determine the appropriate treatment pathway. Prostate volume measurements obtained during the step 120 serve as the foundation for calculating R.I.D. Score values using predetermined factors ranging from 0.0025 to 0.6 injections per cubic centimeter. The assessment process categorizes prostates into distinct size ranges that correspond to specific treatment protocols, with water vapor thermal therapy becoming the selected intervention for prostates measuring between 30 cc and 80 cc in volume.

The step 130 addresses large prostate cases exceeding 80cc in volume, where surgical options include simple prostatectomy procedures performed through open, laparoscopic, or robotic approaches. Holmium Laser Enucleation of the Prostate (HoLEP) and Thulium Laser Enucleation of the Prostate (ThyLEP) represent minimally invasive alternatives within the step 130 protocol for managing enlarged prostate tissue that obstructs urinary flow. The decision-making process incorporates R.I.D. Score calculations even for larger prostates, with score factors preferably ranging from 0.025 to 0.1 injections per cubic centimeter and more preferably from 0.045 to 0.055 injections per cubic centimeter. Following evaluation in the step 130, the process advances to the step 140 for implementation of water vapor thermal therapy when anatomical conditions support this treatment approach.

With continued reference to FIG. 1, the step 140 encompasses multiple treatment modalities for average-sized prostates, including robotic water jet ablation, photoselective vaporization of the prostate, prostatic urethral lift, and transurethral procedures. Water vapor thermal therapy within the step 140 utilizes R.I.D. Score calculations based on prostate volume measurements obtained through Computed Tomography Scan, Magnetic Resonance Imaging, or Ultrasound techniques. The R.I.D. Score factor calculation involves dividing the average number of WVTT injections by the average prostate volume, establishing a standardized approach for treatment planning. Clinical validation demonstrates applicability to prostates with volumes ranging from 18 cc to 93 cc, with a tested R.I.D. Score factor value of 0.05 injections per cubic centimeter providing effective treatment outcomes.

The step 150 addresses small prostate cases measuring less than 30 cc, where treatment options include transurethral incision of the prostate (TUIP) alongside other minimally invasive procedures. R.I.D. Score adjustments account for prostate anatomical variations through calculations that incorporate individual patient characteristics beyond basic volume measurements. The R.I.D.+Score represents an enhanced calculation method that incorporates additional factors related to prostate anatomical variations, providing personalized treatment planning for patients with unique prostatic configurations. System implementation includes an imaging device configured to obtain prostate volume measurements and a processor configured to calculate R.I.D. Score values based on measured volumes and predetermined score factors.

As shown in FIG. 2, the anatomical framework for water vapor thermal therapy involves the bladder 210 positioned above the prostate 250, with the urethra 240 providing access for the delivery device 260. The prostate tissue 230 receives treatment through controlled delivery of a steam 220 generated from water vapor heated to approximately 103 degrees Celsius during WVTT procedures. Each WVTT injection delivers approximately 0.5 mL of water vapor over 9 seconds, with the total number of injections determined through R.I.D. Score calculations rather than traditional measurement protocols. The delivery device 260 eliminates the need to measure 1 cm increments from the bladder neck during procedures and reduces the need to enter the bladder during WVTT procedures, thereby streamlining treatment delivery and reducing procedural complexity.

Referring to FIG. 3, anatomical measurements include the prostatic urethra 310 extending from the bladder region to the distal urethra 330, with the initial pressure point 320 and the secondary pressure point 340 marking measurement locations for treatment planning. The urethral diameter 350 and the urethral angle 360 provide geometric parameters that influence R.I.D. Score adjustments and treatment effectiveness predictions. The normal prostatic urethral angle measures 35 degrees, and deviations from this measurement serve to predict procedure discomfort levels during water vapor delivery. The prostatic urethral angle measurement correlates with patient comfort during treatment, with larger angles typically associated with increased procedural discomfort as demonstrated through clinical correlation studies.

With reference to FIG. 4, the prostate 250 demonstrates the urethral angle 360 measurement used in comprehensive treatment planning calculations that extend beyond basic volume determinations. The imaging device encompasses at least one of a Computed Tomography scanner, a Magnetic Resonance Imaging device, and an Ultrasound device, providing flexibility in clinical implementation across different healthcare settings. Ultrasound imaging utilizes a transrectal probe at 7.5 MHz frequency for accurate volume determination and anatomical assessment. The non-transitory computer-readable medium stores instructions that, when executed by a processor, cause the processor to perform the method for determining WVTT injections based on calculated R.I.D. Score values and anatomical considerations.

As illustrated in FIG. 5A, injection site planning involves the first injection site 510, the second injection site 520, the third injection site 530, and the fourth injection site 540 positioned along the prostatic urethra according to R.I.D. Score calculations. The prostatic angle 550 influences injection placement and treatment effectiveness, with R.I.D. Score adjustments accounting for these spatial relationships through volume-based calculations that predict the total number of injection sites needed for effective treatment. FIGS. 5B-5D demonstrate additional imaging perspectives of injection sites that illustrate the three-dimensional nature of prostate anatomy considered in R.I.D. Score calculations and treatment planning protocols. The systematic approach reduces the risk of acute urinary retention from 11-32% observed with standard protocols to 0% through more precise injection number determination based on individual patient anatomy and calculated density factors.

Referring to FIG. 2, the cross-sectional anatomy during water vapor thermal therapy demonstrates the spatial relationships between the bladder 210, the prostate 250, and the urethra 240 within the treatment configuration. The bladder 210 occupies the superior anatomical position relative to the prostate 250, establishing the foundational structure for urinary flow and treatment access. The prostate tissue 230 surrounds the urethra 240 in a circumferential arrangement, creating the target zone for therapeutic intervention through controlled steam 220 delivery. The delivery device 260 positions within the urethra 240 to provide direct access to the prostate tissue 230 without requiring surgical incision or external approach methods. The steam 220 represents water vapor heated to approximately 103 degrees Celsius, with each WVTT injection delivering approximately 0.5 mL of water vapor over 9 seconds through the delivery device 260 into the surrounding prostate tissue 230.

With continued reference to FIG. 2, the delivery device 260 functions as the primary conduit for therapeutic steam 220 distribution throughout the prostate tissue 230 during treatment procedures. The positioning of the delivery device 260 within the urethra 240 eliminates the need to measure 1 cm increments from the bladder neck during procedures and reduces the need to enter the bladder 210 during WVTT procedures. The steam 220 disperses from the delivery device 260 into the prostate tissue 230 according to calculated injection patterns based on R.I.D. Score determinations. The R.I.D. Score factor ranges from 0.0025 to 0.6 injections per cubic centimeter, with preferred ranges of 0.025 to 0.1 injections per cubic centimeter and more preferred ranges of 0.045 to 0.055 injections per cubic centimeter. The tested value of 0.05 injections per cubic centimeter provides standardized treatment parameters for the delivery device 260 operation within the anatomical configuration shown.

As shown in FIG. 3, the anatomical structure extends from the bladder region through the prostatic urethra 310 to the distal urethra 330, establishing the complete pathway for the delivery device 260 positioning and steam 220 distribution. The initial pressure point 320 and the secondary pressure point 340 define measurement locations along the urethral pathway that influence treatment planning and delivery device 260 positioning. The urethral diameter 350 determines the spatial constraints for the delivery device 260 insertion and operation within the anatomical structure. The urethral angle 360 represents the geometric relationship between the prostatic urethra 310 and the distal urethra 330, with the normal prostatic urethral angle measuring 35 degrees. The prostatic urethral angle measurement serves to predict procedure discomfort levels during delivery device 260 insertion and steam 220 delivery through the anatomical pathway.

Referring to FIG. 4, the prostate 250 demonstrates the urethral angle 360 measurement within the anatomical context of treatment delivery. The urethral angle 360 influences the positioning requirements for the delivery device 260 and affects the distribution patterns of the steam 220 within the prostate tissue 230. The R.I.D. Score calculation incorporates the urethral angle 360 measurements through adjustments that account for individual patient prostate anatomical variations. The R.I.D.+Score incorporates additional factors related to prostate anatomical variations beyond basic volume measurements, including the urethral angle 360 and other geometric parameters. The system includes an imaging device configured to obtain prostate volume measurements and a processor configured to calculate R.I.D. Score values based on anatomical measurements including the urethral angle 360 shown in the prostate 250.

As illustrated in FIG. 5A, the injection site configuration involves the first injection site 510, the second injection site 520, the third injection site 530, and the fourth injection site 540 positioned along the prostatic urethra according to anatomical structure requirements. The prostatic angle 550 influences the spatial distribution of injection sites and affects the delivery device 260 positioning during steam 220 delivery at each location. The first injection site 510 through the fourth injection site 540 represent discrete locations where the delivery device 260 delivers steam 220 into the prostate tissue 230 according to R.I.D. Score calculations. The prostatic angle 550 creates geometric constraints that influence the delivery device 260 approach angles and steam 220 distribution patterns within the prostate tissue 230. FIGS. 5B-5D demonstrate additional anatomical perspectives of injection sites that illustrate the three-dimensional relationships between the delivery device 260 positioning and the prostate tissue 230 structure during treatment delivery.

With reference to FIG. 6, the clinical validation process involves the first step 610, the second step 620, and the third step 630 that establish anatomical measurement protocols for delivery device 260 positioning and steam 220 distribution calculations. The first step 610 encompasses single-surgeon experience with anatomical assessment procedures conducted between October 2023 and June 2024 using standardized delivery device 260 positioning protocols. The second step 620 details patient exclusion criteria based on anatomical variations that affect delivery device 260 insertion or steam 220 distribution patterns. The third step 630 involves anatomical measurement using 7.5 MHz ultrasound with a transrectal probe for accurate prostate volume determination and delivery device 260 positioning guidance. The imaging device includes at least one of a Computed Tomography scanner, a Magnetic Resonance Imaging device, and an Ultrasound device for obtaining prostate volume measurements that guide delivery device 260 positioning and steam 220 distribution calculations.

Referring to FIG. 7A, the symptom scoresheet 700 provides assessment tools for evaluating anatomical function following delivery device 260 treatment and steam 220 distribution within the prostate tissue 230. The symptom scoresheet 700 contains multiple questions related to urinary function that correlate with anatomical changes resulting from steam 220 treatment of the prostate tissue 230. FIG. 7B depicts pain assessment scales that correlate with anatomical factors including the urethral angle 360 measurements and delivery device 260 insertion requirements. The pain assessment scales range from 0 to 10 and correspond to anatomical variations that affect delivery device 260 positioning and patient comfort during steam 220 delivery procedures. The prostatic urethral angle measurement serves to predict procedure discomfort levels based on anatomical geometry and delivery device 260 insertion angles required for effective steam 220 distribution.

As shown in FIG. 8, statistical analysis demonstrates relationships between anatomical measurements and treatment outcomes following delivery device 260 procedures and steam 220 distribution within the prostate tissue 230. The scatter plot displays data points correlating anatomical volume measurements with symptom improvement following steam 220 treatment delivered through the delivery device 260. FIG. 9 presents the graph 900 showing similar anatomical correlations at extended time periods, with the graph 900 displaying relationships between prostate volume measurements and sustained anatomical function improvements following delivery device 260 treatment. The R.I.D. Score factor calculation involves dividing the average number of WVTT injections by the average prostate volume, establishing standardized relationships between anatomical measurements and delivery device 260 treatment parameters.

With continued reference to FIGS. 10, 11, 12, and 13, anatomical function improvements demonstrate the effectiveness of delivery device 260 positioning and steam 220 distribution based on calculated anatomical parameters. The baseline anatomical function measurements show symptom scores of approximately 22.56±3.70, with significant improvements following delivery device 260 treatment and steam 220 distribution within the prostate tissue 230. The anatomical improvements progress from baseline through 4-week and 4-month measurements, demonstrating sustained benefits from delivery device 260 treatment protocols. The statistical significance indicators show P-values of less than 0.0001 for anatomical function improvements, establishing the effectiveness of calculated delivery device 260 positioning and steam 220 distribution based on R.I.D. Score determinations. The anatomical volume measurements range from 18 cc to 93 cc based on clinical study data, demonstrating the broad applicability of delivery device 260 treatment across different anatomical configurations.

As illustrated in FIGS. 14 and 15, pain assessment charts demonstrate relationships between anatomical measurements and patient comfort during delivery device 260 insertion and steam 220 delivery procedures. The charts categorize discomfort levels based on anatomical factors including the urethral angle 360 and delivery device 260 insertion requirements within the prostatic urethra 310 and the distal urethra 330. FIG. 16 shows correlations between anatomical angle measurements and patient discomfort during delivery device 260 positioning, with statistical analysis indicating relationships described by linear equations correlating anatomical geometry with procedural comfort. FIG. 17 presents the procedure discomfort chart 980 showing relationships between anatomical measurements and patient comfort scores during delivery device 260 insertion and steam 220 delivery, including statistical data correlating anatomical variations with procedural discomfort levels. The non-transitory computer-readable medium stores instructions that, when executed by a processor, cause the processor to perform calculations incorporating anatomical measurements for determining delivery device 260 positioning and steam 220 distribution parameters based on individual patient anatomical configurations.

Referring to FIG. 3, the prostatic urethra 310 extends from the bladder region through a curved pathway to connect with the distal urethra 330, establishing the anatomical framework for precise measurement protocols during water vapor thermal therapy planning. The initial pressure point 320 and the secondary pressure point 340 define specific measurement locations along the urethral pathway that provide quantitative data for treatment parameter calculations. The urethral diameter 350 establishes dimensional constraints that influence delivery device positioning and steam distribution patterns within the anatomical structure. The urethral angle 360 represents the geometric relationship between the prostatic urethra 310 and the distal urethra 330, with this angular measurement serving as a fundamental parameter for predicting procedural outcomes and patient comfort levels during treatment delivery.

The normal prostatic urethral angle measures 35 degrees, providing a baseline reference for anatomical assessment and treatment planning calculations. Deviations from this standard angular measurement correlate directly with procedural discomfort levels and influence the positioning requirements for therapeutic intervention. The prostatic urethral angle measurement functions as a predictive tool for procedure discomfort levels, with larger angles typically associated with increased patient discomfort during delivery device insertion and steam delivery procedures. The angular measurement system incorporates geometric calculations that account for individual patient anatomical variations and influence R.I.D. Score adjustments through enhanced calculation methods.

With reference to FIG. 4, the prostate 250 demonstrates the urethral angle 360 measurement within the context of comprehensive anatomical assessment protocols. The urethral angle 360 measurement integrates with volume-based calculations to provide enhanced treatment planning that extends beyond basic dimensional assessments. The R.I.D. Score calculation incorporates the urethral angle 360 measurements through systematic adjustments that account for individual patient prostate anatomical variations and geometric constraints. The R.I.D.+Score incorporates additional factors related to prostate anatomical variations, including the urethral angle 360 and other geometric parameters that influence treatment effectiveness and patient comfort predictions.

As shown in FIG. 16, the correlation between prostatic urethral angles and numeric pain rating scale scores demonstrates quantitative relationships between anatomical measurements and patient discomfort during procedures. The statistical analysis indicates a positive correlation described by the linear equation y=0.0436x+1.3003, establishing mathematical relationships between anatomical geometry and procedural comfort levels. The correlation data includes statistical parameters with a standard coefficient of 0.328, p-value of 0.019, and an R² value of 0.1076, providing quantitative validation for the predictive capabilities of angular measurements in treatment planning. The prostatic urethral angle measurements range across different patient populations, with each measurement contributing to individualized treatment parameter calculations and comfort prediction protocols.

Referring to FIG. 17, the procedure discomfort chart 980 presents comprehensive statistical analysis of the relationship between NPRS-11 scores and prostatic urethral angle measurements. The procedure discomfort chart 980 includes coefficients and statistical data with standardized and unstandardized coefficients, standard error values, and confidence intervals that establish quantitative relationships between anatomical measurements and patient comfort predictions. The statistical analysis within the procedure discomfort chart 980 provides validation for angular measurement protocols and their integration into R.I.D. Score calculation systems. The data demonstrates that prostatic urethral angle measurements function as reliable predictors for procedural discomfort levels, enabling healthcare providers to implement appropriate comfort management protocols based on individual patient anatomical assessments.

With continued reference to FIG. 3, the initial pressure point 320 and the secondary pressure point 340 establish measurement locations that correspond to specific anatomical landmarks along the urethral pathway. The pressure point measurements provide quantitative data that correlates with steam distribution patterns and treatment effectiveness parameters during water vapor thermal therapy procedures. The urethral diameter 350 measurements at these pressure points influence the spatial constraints for delivery device operation and steam distribution calculations. The geometric relationships between the initial pressure point 320, the secondary pressure point 340, and the urethral angle 360 create a comprehensive measurement framework that supports precise treatment parameter calculations and procedural planning protocols.

As illustrated in FIG. 5A, the prostatic angle 550 influences the spatial distribution of injection sites and affects delivery device positioning during steam delivery at each treatment location. The prostatic angle 550 creates geometric constraints that influence delivery device approach angles and steam distribution patterns within the prostate tissue 230 during therapeutic intervention. The angular measurements including the prostatic angle 550 integrate with the first injection site 510, the second injection site 520, the third injection site 530, and the fourth injection site 540 positioning calculations to optimize treatment delivery patterns. The prostatic angle 550 measurements contribute to R.I.D. Score adjustments that account for individual patient anatomical variations and geometric constraints that influence treatment effectiveness and procedural comfort levels.

Referring to FIG. 6, the third step 630 involves anatomical measurement protocols using 7.5 MHz ultrasound with a transrectal probe for accurate angular assessment and dimensional analysis. The ultrasound imaging protocols provide precise measurements of prostatic urethral angles and dimensional parameters that support R.I.D. Score calculations and treatment planning procedures. The imaging device includes at least one of a Computed Tomography scanner, a Magnetic Resonance Imaging device, and an Ultrasound device for obtaining comprehensive anatomical measurements that guide angular analysis and treatment parameter calculations. The measurement protocols established in the third step 630 provide standardized approaches for angular assessment across different patient populations and clinical settings, ensuring consistent data collection for R.I.D. Score calculation systems.

With reference to FIGS. 14 and 15, pain assessment charts demonstrate the practical application of angular measurements in predicting patient comfort during water vapor thermal therapy procedures. The charts categorize discomfort levels as mild (0-3), moderate (4-6), and severe (7-10) on numerical rating scales that correspond to specific ranges of prostatic urethral angle measurements. The pain assessment protocols integrate angular measurement data with patient comfort predictions, enabling healthcare providers to implement appropriate procedural modifications based on individual anatomical assessments. The relationship between angular measurements and comfort predictions supports the development of personalized treatment protocols that account for individual patient anatomical variations and geometric constraints.

Referring to FIG. 5A, the first injection site 510, the second injection site 520, the third injection site 530, and the fourth injection site 540 establish a systematic spatial arrangement along the prostatic urethra for water vapor thermal therapy delivery. The first injection site 510 through the fourth injection site 540 represent discrete anatomical locations where controlled steam delivery occurs according to R.I.D. Score calculations that account for prostate volume measurements and anatomical variations. The prostatic angle 550 influences the geometric positioning of each injection site and affects the spatial distribution patterns of water vapor within the prostate tissue 230 during therapeutic intervention. The injection site configuration eliminates the need to measure 1 cm increments from the bladder neck during the procedure, instead relying on calculated positioning based on prostate volume measurements obtained through Computed Tomography Scan, Magnetic Resonance Imaging, or Ultrasound techniques. The spatial arrangement of the first injection site 510 through the fourth injection site 540 accounts for individual patient anatomical variations through R.I.D. Score adjustments that incorporate the prostatic angle 550 measurements and other geometric parameters affecting treatment delivery.

With continued reference to FIG. 5A, the prostatic angle 550 creates geometric constraints that influence the delivery device 260 approach angles and steam 220 distribution patterns at each injection site location. The prostatic angle 550 measurements contribute to R.I.D.+Score calculations that incorporate additional factors related to prostate anatomical variations beyond basic volume determinations. The first injection site 510 positioning accounts for the prostatic angle 550 geometry and establishes the initial treatment location along the prostatic urethra pathway. The second injection site 520 through the fourth injection site 540 follow calculated spacing intervals based on R.I.D. Score factors ranging from 0.0025 to 0.6 injections per cubic centimeter, with preferred ranges of 0.025 to 0.1 injections per cubic centimeter and more preferred ranges of 0.045 to 0.055 injections per cubic centimeter. The tested value of 0.05 injections per cubic centimeter provides standardized positioning parameters for the first injection site 510 through the fourth injection site 540 configuration within the anatomical structure shown.

As illustrated in FIGS. 5B-5D, magnetic resonance imaging visualization provides detailed cross-sectional views of injection sites within the prostate tissue 230 and surrounding anatomical structures. The magnetic resonance imaging scans demonstrate different stages and locations of water vapor thermal therapy injections, with each scan showing varying levels of contrast that visualize the prostate tissue 230 structure and targeted treatment areas. The imaging visualization supports the spatial arrangement calculations for the first injection site 510 through the fourth injection site 540 by providing three-dimensional anatomical data that accounts for individual patient prostate configurations. The magnetic resonance imaging data integrates with R.I.D. Score calculations to establish precise injection site positioning that accounts for the prostatic angle 550 and other anatomical variations affecting treatment delivery patterns. The cross-sectional imaging perspectives demonstrate the relationship between calculated injection site positions and actual anatomical structures, validating the spatial distribution patterns established through R.I.D. Score factor calculations.

Referring to FIG. 2, the anatomical framework supporting injection site planning involves the prostate tissue 230 surrounding the urethra 240, with the delivery device 260 providing access for steam 220 distribution at calculated injection locations. The prostate tissue 230 receives therapeutic intervention through controlled delivery of steam 220 heated to approximately 103 degrees Celsius, with each WVTT injection delivering approximately 0.5 mL of water vapor over 9 seconds at designated injection sites. The delivery device 260 positioning within the urethra 240 enables precise steam 220 delivery to the first injection site 510 through the fourth injection site 540 without requiring surgical incision or external approach methods. The spatial relationship between the bladder 210, the prostate 250, and the urethra 240 establishes the anatomical constraints that influence injection site positioning and steam 220 distribution patterns during treatment procedures. The delivery device 260 operation reduces the need to enter the bladder 210 during WVTT procedures, streamlining treatment delivery through calculated injection site positioning based on R.I.D. Score determinations.

With reference to FIG. 3, the prostatic urethra 310 extends through the anatomical pathway that contains the first injection site 510 through the fourth injection site 540 positioning locations. The urethral angle 360 between the prostatic urethra 310 and the distal urethra 330 influences the geometric calculations for injection site spacing and steam distribution patterns within the prostate tissue 230. The normal prostatic urethral angle of 35 degrees provides baseline reference measurements for injection site positioning calculations, with deviations from this standard measurement affecting the spatial arrangement of the first injection site 510 through the fourth injection site 540. The prostatic urethral angle measurement functions as a predictive tool for procedure discomfort levels during injection site treatment delivery, with larger angles typically associated with increased procedural complexity and modified injection site positioning requirements. The urethral diameter 350 establishes dimensional constraints that influence delivery device 260 positioning and steam 220 distribution calculations at each injection site location along the prostatic urethra 310 pathway.

As shown in FIG. 4, the prostate 250 demonstrates the urethral angle 360 measurement within the context of injection site planning and spatial distribution calculations. The urethral angle 360 measurement integrates with volume-based R.I.D. Score calculations to establish enhanced injection site positioning that extends beyond basic dimensional assessments. The imaging device includes at least one of a Computed Tomography scanner, a Magnetic Resonance Imaging device, and an Ultrasound device for obtaining comprehensive anatomical measurements that guide injection site positioning and prostatic angle 550 analysis. The system includes an imaging device configured to obtain prostate volume measurements and a processor configured to calculate R.I.D. Score values based on anatomical measurements including the urethral angle 360 and injection site positioning requirements. The processor calculations account for the urethral angle 360 measurements through systematic adjustments that determine optimal spacing for the first injection site 510 through the fourth injection site 540 configuration.

Referring to FIG. 6, the third step 630 involves anatomical measurement protocols using 7.5 MHz ultrasound with a transrectal probe for accurate injection site positioning and prostatic angle 550 assessment. The ultrasound imaging protocols provide precise measurements that support injection site spacing calculations and treatment planning procedures for the first injection site 510 through the fourth injection site 540 configuration. The measurement protocols established in the third step 630 provide standardized approaches for injection site assessment across different patient populations with prostate volumes ranging from 18 cc to 93 cc based on clinical study data. The imaging data collection supports R.I.D. Score factor calculations that determine injection site positioning, with the R.I.D. Score factor calculated by dividing the average number of WVTT injections by the average prostate volume. The standardized measurement approach ensures consistent injection site positioning data for R.I.D. Score calculation systems across different clinical settings and patient anatomical configurations.

With continued reference to FIGS. 5B-5D, the magnetic resonance imaging visualization demonstrates the three-dimensional spatial relationships between calculated injection site positions and actual prostate tissue 230 structures during treatment delivery. The cross-sectional imaging views provide detailed anatomical information that validates the positioning calculations for the first injection site 510 through the fourth injection site 540 within individual patient prostate configurations. The magnetic resonance imaging data supports R.I.D. Score adjustments that account for prostate anatomical variations through enhanced calculation methods that incorporate the prostatic angle 550 and other geometric parameters. The imaging visualization confirms the effectiveness of calculated injection site spacing in achieving comprehensive steam 220 distribution throughout the prostate tissue 230 while maintaining procedural safety and treatment efficacy. The non-transitory computer-readable medium stores instructions that, when executed by a processor, cause the processor to perform injection site positioning calculations incorporating magnetic resonance imaging data and anatomical measurements for determining optimal treatment delivery parameters based on individual patient prostate configurations.

As illustrated in FIG. 16, the correlation between prostatic urethral angles and procedural discomfort demonstrates the importance of angular measurements in injection site planning and treatment delivery protocols. The statistical analysis establishes quantitative relationships between anatomical geometry and procedural comfort levels during injection site treatment, with the linear equation y=0.0436x+1.3003 describing the mathematical relationship between angular measurements and patient discomfort predictions. The prostatic urethral angle measurements influence injection site positioning requirements and affect the delivery device 260 approach angles needed for effective steam 220 distribution at the first injection site 510 through the fourth injection site 540. The correlation data supports the integration of angular measurements into R.I.D. Score calculations that determine injection site spacing and treatment delivery parameters. The statistical validation demonstrates that injection site planning protocols incorporating prostatic urethral angle measurements reduce the risk of acute urinary retention from 11-32% observed with standard protocols to 0% through more precise positioning calculations and anatomical assessment procedures.

Referring to FIG. 6, the clinical validation process establishes comprehensive methodology for R.I.D. Score formula development through systematic patient selection and data collection protocols. The first step 610 encompasses single-surgeon experience with procedures conducted between October 2023 and June 2024, establishing standardized treatment protocols across a defined temporal period. The cohort characteristics include 60 patients with mean age of 67.4±6.62 years, prostate volume of 54.86±25.40 cc, PSA levels of 2.38±2.38 ng/mL, and prostatic urethral angle measurements of 39.43±10.41 degrees. The patient population demonstrates prostate volumes ranging from 18 cc to 93 cc based on clinical study data, establishing the broad applicability of R.I.D. Score calculations across diverse anatomical configurations. The single-surgeon approach eliminates procedural variability and ensures consistent treatment delivery protocols throughout the study period.

The second step 620 details patient exclusion criteria that affect data integrity and treatment outcome measurements within the clinical validation process. Four patients representing specific exclusion categories were removed from the study cohort based on non-standard pain protocol implementation and chronic Foley catheter usage patterns. The exclusion criteria account for variables that could influence R.I.D. Score factor calculations and treatment outcome assessments. Patients with non-standard pain protocols were excluded to maintain consistency in discomfort measurement and procedural assessment protocols. Chronic Foley catheter users were excluded due to altered baseline urinary function parameters that could affect post-treatment outcome measurements and statistical analysis validity.

With continued reference to FIG. 6, the third step 630 involves comprehensive measurement and analysis protocols using 7.5 MHz ultrasound with a transrectal probe for accurate prostate volume determination and anatomical assessment. The ultrasound imaging specifications provide precise volumetric measurements that serve as the foundation for R.I.D. Score factor calculations ranging from 0.0025 to 0.6 injections per cubic centimeter. The transrectal probe positioning enables detailed visualization of the prostate tissue 230 and surrounding anatomical structures for comprehensive volume assessment. Linear regression models and paired T-tests were conducted using SPSS software to establish statistical relationships between prostate volume measurements and treatment outcomes. The statistical analysis incorporates AUA SS and NPRS-11 metrics to quantify treatment effectiveness and patient comfort correlations with R.I.D. Score calculations.

As shown in FIG. 7A, the symptom scoresheet 700 provides standardized assessment tools for evaluating treatment outcomes and validating R.I.D. Score effectiveness across the patient cohort. The symptom scoresheet 700 contains multiple questions related to urinary function with corresponding numerical rating scales ranging from 0 to 5, enabling quantitative assessment of symptom frequency over specified time periods. The scoresheet evaluation includes sections for incomplete emptying, frequency, intermittency, urgency, weak stream, straining, and nocturia symptoms that correlate with prostate volume measurements and R.I.D. Score calculations. FIG. 7B depicts the pain assessment scale ranging from 0 to 10 with gradations from “No pain” to “Worst possible pain” that correlate with the prostatic urethral angle measurements for predicting procedure discomfort levels. The normal prostatic urethral angle of 35 degrees provides baseline reference measurements, with deviations affecting discomfort predictions and R.I.D. Score adjustments.

Referring to FIG. 8, statistical analysis demonstrates quantitative relationships between R.I.D. Score values and symptom improvement at 4 weeks post-treatment through scatter plot visualization and linear regression analysis. The graph displays data points representing the relationship between injections per milliliter of prostate volume on the x-axis and AUA SS change values on the y-axis. The linear regression line shows the equation y=36.078x+13.751 with an R² value of 0.022, establishing mathematical relationships between R.I.D. Score factors and treatment outcomes. The x-axis ranges from 0.0000 to 0.1200 injections/mL of prostate volume, encompassing the preferred R.I.D. Score factor range of 0.025 to 0.1 injections per cubic centimeter and the more preferred range of 0.045 to 0.055 injections per cubic centimeter. The statistical correlation validates the effectiveness of R.I.D. Score factor calculations in predicting treatment outcomes across the patient cohort.

With reference to FIG. 9, the graph 900 presents extended statistical analysis showing R.I.D. Score versus AUA SS change at 4 months post-treatment, demonstrating sustained treatment effectiveness over extended time periods. The graph 900 displays scatter plot data with a linear regression line where the x-axis represents injections/mL of prostate volume and the y-axis represents AUA SS change values. The linear equation y=13.926x+17.844 with an R² value of 0.0045 establishes long-term correlation patterns between R.I.D. Score calculations and sustained symptom improvement. The tested value of 0.05 injections per cubic centimeter demonstrates consistent effectiveness across both 4-week and 4-month measurement periods. The statistical analysis supports the R.I.D. Score factor calculation method involving dividing the average number of WVTT injections by the average prostate volume to establish standardized treatment parameters.

As illustrated in FIGS. 10, 11, 12, and 13, treatment outcome data demonstrates comprehensive symptom score improvements over time following WVTT procedures using R.I.D. Score calculations across the clinical cohort. The baseline measurements show symptom scores of approximately 22.56±3.70, with significant reductions to approximately 7 at 4 weeks and further reductions to approximately 4 at 4 months post-treatment. The statistical significance indicators show P-values of less than 0.0001 for both 4-week and 4-month measurements, establishing the effectiveness of R.I.D. Score-based treatment planning across the patient population. The data demonstrates that R.I.D. Score adjustments account for prostate anatomical variations through enhanced calculation methods that incorporate individual patient characteristics. The R.I.D.+Score incorporates additional factors related to prostate anatomical variations beyond basic volume measurements, providing personalized treatment planning for patients with diverse prostatic configurations.

Referring to FIG. 16, the correlation between prostatic urethral angles and numeric pain rating scale scores establishes quantitative relationships between anatomical measurements and procedural discomfort across the clinical cohort. The statistical analysis indicates a positive correlation described by the linear equation y=0.0436x+1.3003, with a standard coefficient of 0.328, p-value of 0.019, and an R² value of 0.1076. The prostatic urethral angle measurement functions as a predictive tool for procedure discomfort levels, with larger angles associated with increased patient discomfort during delivery device insertion and steam delivery procedures. The correlation data supports the integration of angular measurements into R.I.D. Score calculations that determine treatment delivery parameters and patient comfort predictions. The statistical validation demonstrates that treatment planning protocols incorporating prostatic urethral angle measurements reduce the risk of acute urinary retention from 11-32% observed with standard protocols to 0% through precise anatomical assessment procedures.

With continued reference to FIG. 17, the procedure discomfort chart 980 presents comprehensive statistical analysis of the relationship between NPRS-11 scores and prostatic urethral angle measurements across the clinical validation cohort. The procedure discomfort chart 980 includes coefficients and statistical data with standardized and unstandardized coefficients, standard error values, and confidence intervals that establish quantitative relationships between anatomical measurements and patient comfort predictions. The statistical analysis validates the effectiveness of ultrasound imaging performed using a transrectal probe at 7.5 MHz frequency for accurate anatomical assessment and discomfort prediction protocols. The data collection methodology eliminates the need to measure 1 cm increments from the bladder neck during procedures and reduces the need to enter the bladder during WVTT procedures through calculated positioning based on imaging-derived measurements. The non-transitory computer-readable medium stores instructions that, when executed by a processor, cause the processor to perform R.I.D. Score calculations incorporating clinical validation data and anatomical measurements for determining treatment delivery parameters based on individual patient configurations within the validated cohort parameters.

Referring to FIG. 7A, the symptom scoresheet 700 establishes a comprehensive evaluation framework for quantifying urinary symptoms and treatment outcomes in patients undergoing water vapor thermal therapy procedures. The symptom scoresheet 700 contains multiple questions related to urinary function with corresponding numerical rating scales ranging from 0 to 5, where patients indicate the frequency of various urinary symptoms experienced over the past month. The scoresheet evaluation encompasses sections for incomplete emptying, frequency, intermittency, urgency, weak stream, straining, and nocturia symptoms that correlate directly with prostate volume measurements and R.I.D. Score calculations. The numerical rating system within the symptom scoresheet 700 provides quantitative data for statistical analysis and treatment outcome assessment across patient populations with prostate volumes ranging from 18 cc to 93 cc based on clinical study data. The symptom scoresheet 700 functions as a standardized measurement tool that eliminates subjective assessment variability and enables precise quantification of treatment effectiveness following R.I.D. Score-based injection planning.

With reference to FIG. 7B, the pain assessment scale establishes a numerical rating system ranging from 0 to 10 with specific gradations that correspond to increasing levels of patient discomfort during water vapor thermal therapy procedures. The scale presents a horizontal gradient that transitions from “No pain” at the 0 mark through “Moderate pain” at the midpoint to “Worst possible pain” at the 10 mark, providing clear reference points for patient comfort assessment. The numerical pain rating scale correlates directly with prostatic urethral angle measurements for predicting procedure discomfort levels during delivery device insertion and steam delivery procedures. The normal prostatic urethral angle of 35 degrees provides baseline reference measurements, with deviations from this standard measurement affecting discomfort predictions and requiring R.I.D. Score adjustments through enhanced calculation methods. The pain assessment scale integrates with the prostatic urethral angle measurement system to predict procedure discomfort levels, with larger angles typically associated with increased patient discomfort during therapeutic intervention.

As shown in FIG. 14, the pain score rating chart demonstrates the practical application of numerical assessment tools in clinical settings for water vapor thermal therapy procedures. The chart displays a numerical scale from 0 to 10 with a circular indicator positioned at approximately level 3, illustrating the measurement methodology used during patient assessment protocols. The scale incorporates a gradient coloring scheme that transitions from light gray at the low end to black at the high end of the pain scale, providing visual reference points that correspond to increasing discomfort levels. The pain score rating chart functions in conjunction with the symptom scoresheet 700 to provide comprehensive patient assessment data that supports R.I.D. Score calculations and treatment planning protocols. The measurement system accounts for individual patient anatomical variations through the prostatic urethral angle measurement system, which serves to predict procedure discomfort levels and guide appropriate comfort management protocols.

Referring to FIG. 15, the enhanced pain score rating chart presents a categorized assessment system that divides patient discomfort into three distinct ranges: mild discomfort ranging from 0-3, moderate discomfort ranging from 4-6, and pain ranging from 7-10. The chart includes color-coded indicators with yellow, orange, and red circles containing numerical values of 38, 13, and 1 respectively, positioned along the scale to represent patient distribution patterns across discomfort categories. The categorized system provides healthcare providers with standardized reference points for implementing appropriate procedural modifications based on predicted discomfort levels derived from prostatic urethral angle measurements. The pain assessment categories correlate with R.I.D. Score adjustments that account for prostate anatomical variations through calculations incorporating the prostatic urethral angle and other geometric parameters affecting treatment delivery. The measurement system supports the method that reduces the risk of acute urinary retention from 11-32% to 0% compared to standard protocols through precise anatomical assessment and comfort prediction protocols.

With continued reference to FIG. 16, the correlation between prostatic urethral angles and numeric pain rating scale scores establishes quantitative relationships between anatomical measurements and procedural discomfort across clinical patient populations. The statistical analysis indicates a positive correlation described by the linear equation y=0.0436x+1.3003, with a standard coefficient of 0.328, p-value of 0.019, and an R² value of 0.1076 that validates the predictive capabilities of angular measurements in treatment planning. The scatter plot displays data points representing individual patient measurements with a linear regression line demonstrating the mathematical relationship between prostatic urethral angle measurements in degrees and pain ratings on the numeric scale from 0-8. The correlation data supports the integration of angular measurements into R.I.D. Score calculations that determine treatment delivery parameters and patient comfort predictions based on individual anatomical configurations. The statistical validation demonstrates that treatment planning protocols incorporating prostatic urethral angle measurements enable healthcare providers to implement appropriate comfort management strategies based on predicted discomfort levels.

As illustrated in FIG. 17, the procedure discomfort chart 980 presents comprehensive statistical analysis of the relationship between NPRS-11 scores and prostatic urethral angle measurements across clinical validation cohorts. The procedure discomfort chart 980 includes coefficients and statistical data with standardized and unstandardized coefficients, standard error values, and confidence intervals that establish quantitative relationships between anatomical measurements and patient comfort predictions during water vapor thermal therapy procedures. The statistical analysis validates the effectiveness of ultrasound imaging performed using a transrectal probe at 7.5 MHz frequency for accurate anatomical assessment and discomfort prediction protocols that support R.I.D. Score calculations. The data collection methodology within the procedure discomfort chart 980 eliminates the need to measure 1 cm increments from the bladder neck during procedures and reduces the need to enter the bladder during WVTT procedures through calculated positioning based on imaging-derived measurements. The comprehensive statistical framework supports R.I.D. Score factor calculations ranging from 0.0025 to 0.6 injections per cubic centimeter, with preferred ranges of 0.025 to 0.1 injections per cubic centimeter and more preferred ranges of 0.045 to 0.055 injections per cubic centimeter.

Referring to FIG. 8, the statistical analysis demonstrates quantitative relationships between R.I.D. Score values and symptom improvement measurements at 4 weeks post-treatment through scatter plot visualization and linear regression analysis that validates the effectiveness of the symptom scoresheet 700 assessment protocols. The graph displays data points representing the relationship between injections per milliliter of prostate volume and AUA SS change values derived from the symptom scoresheet 700 evaluations. The linear regression line shows the equation y=36.078x+13.751 with an R² value of 0.022, establishing mathematical relationships between R.I.D. Score factors and treatment outcomes quantified through the symptom scoresheet 700 measurement system. The statistical correlation validates the effectiveness of R.I.D. Score factor calculations in predicting treatment outcomes across patient populations, with the tested value of 0.05 injections per cubic centimeter demonstrating consistent effectiveness in symptom improvement measurements. The data integration between the symptom scoresheet 700 and R.I.D. Score calculations supports treatment planning protocols that account for individual patient anatomical variations through enhanced calculation methods.

With reference to FIG. 9, the graph 900 presents extended statistical analysis showing R.I.D. Score versus AUA SS change at 4 months post-treatment, demonstrating sustained treatment effectiveness over extended time periods as measured through the symptom scoresheet 700 evaluation system. The graph 900 displays scatter plot data with a linear regression line where symptom improvement values derived from the symptom scoresheet 700 correlate with R.I.D. Score calculations across the patient cohort. The linear equation y=13.926x+17.844 with an R² value of 0.0045 establishes long-term correlation patterns between R.I.D. Score calculations and sustained symptom improvement as quantified through standardized assessment protocols. The statistical analysis supports the R.I.D. Score factor calculation method involving dividing the average number of WVTT injections by the average prostate volume to establish standardized treatment parameters that correlate with symptom improvement measurements. The graph 900 validates the integration of the symptom scoresheet 700 assessment data with R.I.D. Score calculations for comprehensive treatment outcome evaluation across extended follow-up periods.

As shown in FIGS. 10, 11, 12, and 13, treatment outcome data demonstrates comprehensive symptom score improvements over time following WVTT procedures using R.I.D. Score calculations, with measurements derived from the symptom scoresheet 700 evaluation protocols across clinical patient populations. The baseline measurements show symptom scores of approximately 22.56±3.70 as quantified through the symptom scoresheet 700 assessment system, with significant reductions to approximately 7 at 4 weeks and further reductions to approximately 4 at 4 months post-treatment. The statistical significance indicators show P-values of less than 0.0001 for both 4-week and 4-month measurements, establishing the effectiveness of R.I.D. Score-based treatment planning as validated through standardized symptom assessment protocols. The data demonstrates that R.I.D. Score adjustments account for prostate anatomical variations through enhanced calculation methods that incorporate individual patient characteristics and correlate with symptom improvement measurements obtained through the symptom scoresheet 700 evaluation system. The comprehensive outcome data supports the integration of pain assessment scales with the symptom scoresheet 700 to provide complete patient evaluation frameworks that guide R.I.D. Score calculations and treatment delivery parameters based on individual anatomical configurations and comfort prediction protocols.

Referring to FIG. 8, the statistical analysis demonstrates quantitative relationships between R.I.D. Score values and symptom improvement measurements at 4 weeks post-treatment through scatter plot visualization and linear regression analysis. The graph displays data points representing the relationship between injections per milliliter of prostate volume on the x-axis and AUA SS change values on the y-axis derived from clinical assessment protocols. The linear regression line shows the equation y=36.078x+13.751 with an R² value of 0.022, establishing mathematical relationships between R.I.D. Score factors and treatment outcomes across the patient population. The x-axis ranges from 0.0000 to 0.1200 injections/mL of prostate volume, encompassing R.I.D. Score factor ranges from 0.0025 to 0.6 injections per cubic centimeter. The statistical correlation validates the effectiveness of R.I.D. Score factor calculations in predicting treatment outcomes, with the tested value of 0.05 injections per cubic centimeter demonstrating consistent effectiveness in symptom improvement measurements across the clinical cohort.

With reference to FIG. 9, the graph 900 presents extended statistical analysis showing R.I.D. Score versus AUA SS change at 4 months post-treatment, demonstrating sustained treatment effectiveness over extended time periods. The graph 900 displays scatter plot data with a linear regression line where the x-axis represents injections/mL of prostate volume and the y-axis represents AUA SS change values measured through standardized assessment protocols. The linear equation y=13.926x+17.844 with an R² value of 0.0045 establishes long-term correlation patterns between R.I.D. Score calculations and sustained symptom improvement across the patient cohort. The statistical analysis supports the R.I.D. Score factor calculation method involving dividing the average number of WVTT injections by the average prostate volume to establish standardized treatment parameters. The graph 900 validates the integration of R.I.D. Score calculations with treatment outcome measurements across extended follow-up periods, demonstrating the sustained effectiveness of calculated injection parameters.

As shown in FIG. 10, treatment outcome data demonstrates comprehensive symptom score improvements over time following WVTT procedures using R.I.D. Score calculations across clinical patient populations. The graph displays three data points along a timeline including baseline, 4 weeks, and 4 months measurements that establish temporal relationships between R.I.D. Score-based treatment delivery and symptom improvement patterns. The baseline measurement shows a symptom score of approximately 22, which decreases to approximately 7 at 4 weeks, and further decreases to approximately 4 at 4 months post-treatment. The graph includes error bars for each measurement point and indicates P-values of less than 0.0001 for both the 4-week and 4-month measurements, establishing statistical significance for treatment effectiveness. The temporal progression demonstrates the sustained effectiveness of R.I.D. Score calculations in achieving progressive symptom improvement across extended treatment periods.

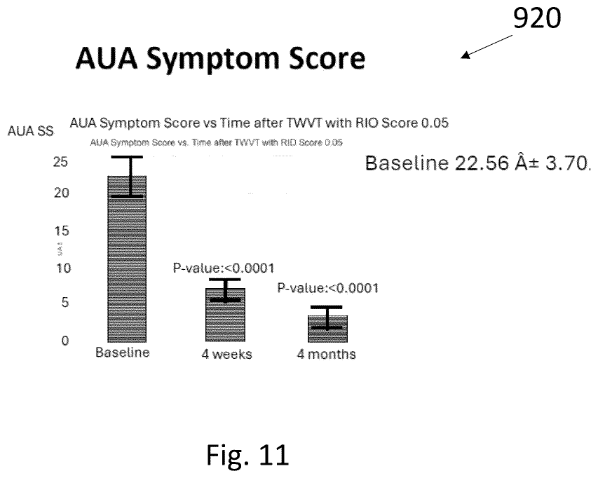

Referring to FIG. 11, the baseline symptom score measurement establishes the initial clinical parameters for R.I.D. Score correlation analysis across the patient population. The graph displays a baseline measurement of 22.56±3.70 with a blue vertical bar representing the baseline score and an orange arrow pointing to the baseline value. The y-axis shows AUA symptom score values while the x-axis indicates the baseline measurement point, with error bars indicating the standard deviation from the baseline measurement. The baseline data provides the foundation for calculating symptom improvement correlations with R.I.D. Score factors ranging from 0.025 to 0.1 injections per cubic centimeter and more preferably from 0.045 to 0.055 injections per cubic centimeter. The standardized baseline measurement enables consistent correlation analysis between R.I.D. Score calculations and treatment outcome assessments across diverse patient anatomical configurations with prostate volumes ranging from 18 cc to 93 cc based on clinical study data.

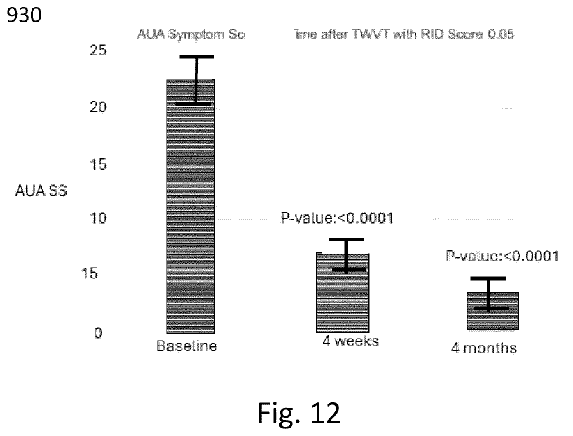

With continued reference to FIG. 12, the statistical analysis demonstrates symptom score improvements at 4 weeks post-treatment using R.I.D. Score calculations with a factor of 0.05 injections per cubic centimeter. The graph displays data points showing symptom score measurements over time with error bars indicating standard deviation values that validate the statistical significance of treatment outcomes. The graph includes baseline measurements and subsequent measurements showing a decrease in symptom scores, with numerical values indicating a difference of 15.50±4.62 and a p-value of less than 0.0001. The measurement at the 4-week mark shows values of 7.06±3.07, demonstrating substantial symptom improvement following R.I.D. Score-based treatment delivery. The statistical validation supports the effectiveness of the tested R.I.D. Score factor value of 0.05 injections per cubic centimeter in achieving clinically significant symptom improvements across the patient population.

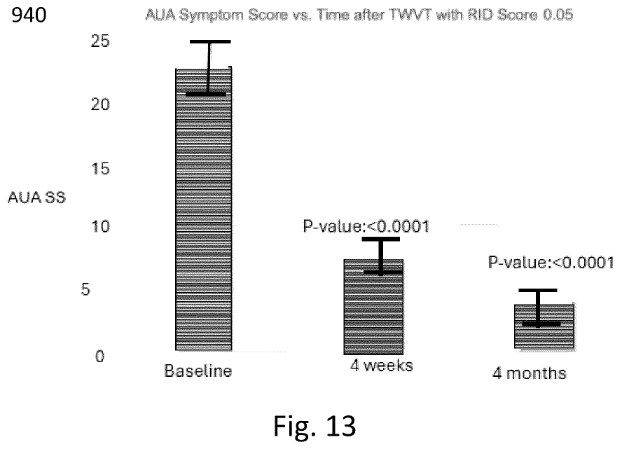

As illustrated in FIG. 13, the extended correlation analysis shows symptom score improvements at 4 months post-treatment using R.I.D. Score calculations with the tested factor of 0.05 injections per cubic centimeter. The graph illustrates changes in AUA symptom scores over time with data points showing a significant decrease in symptom scores from baseline measurements. The vertical axis represents AUA symptom score values while the horizontal axis represents time intervals, with the graph demonstrating a clear downward trend in symptom scores following R.I.D. Score-based treatment delivery. The statistical significance indicators show a p-value of less than 0.0001 with data showing a difference of 18.46±4.04 and a value of 3.91±2.36 at the 4-month time point. The correlation analysis validates the sustained effectiveness of R.I.D. Score calculations in maintaining symptom improvement across extended follow-up periods, demonstrating the long-term benefits of calculated injection parameters.

Referring to FIG. 6, the clinical validation process establishes the methodological framework for R.I.D. Score correlation analysis through systematic patient selection and data collection protocols. The first step 610 encompasses single-surgeon experience with procedures conducted between October 2023 and June 2024, establishing standardized treatment protocols across a defined temporal period for correlation analysis. The cohort characteristics include 60 patients with mean age of 67.4±6.62 years, prostate volume of 54.86±25.40 cc, PSA levels of 2.38±2.38 ng/mL, and prostatic urethral angle measurements of 39.43±10.41 degrees. The second step 620 details patient exclusion criteria affecting correlation analysis integrity, with four patients excluded based on non-standard pain protocol implementation and chronic Foley catheter usage patterns. The third step 630 involves comprehensive measurement protocols using 7.5 MHz ultrasound with a transrectal probe for accurate prostate volume determination that supports R.I.D. Score correlation calculations. Linear regression models and paired T-tests were conducted using SPSS software to establish statistical relationships between prostate volume measurements and treatment outcomes, incorporating AUA SS and NPRS-11 metrics for comprehensive correlation analysis.

With reference to FIG. 7A, the symptom scoresheet 700 establishes the standardized measurement framework for quantifying treatment outcomes in R.I.D. Score correlation analysis. The symptom scoresheet 700 contains multiple questions related to urinary function with corresponding numerical rating scales ranging from 0 to 5, where patients indicate symptom frequency over specified time periods for correlation assessment. The scoresheet evaluation encompasses sections for incomplete emptying, frequency, intermittency, urgency, weak stream, straining, and nocturia symptoms that correlate directly with R.I.D. Score calculations and treatment effectiveness measurements. The numerical rating system within the symptom scoresheet 700 provides quantitative data for statistical analysis and correlation assessment across patient populations with diverse prostate anatomical configurations. FIG. 7B depicts the pain assessment scale ranging from 0 to 10 with gradations from “No pain” to “Worst possible pain” that correlate with prostatic urethral angle measurements and R.I.D. Score adjustments for comprehensive treatment outcome analysis.

As shown in FIG. 16, the correlation between prostatic urethral angles and numeric pain rating scale scores establishes quantitative relationships between anatomical measurements and procedural outcomes that influence R.I.D. Score correlation analysis. The statistical analysis indicates a positive correlation described by the linear equation y=0.0436x+1.3003, with a standard coefficient of 0.328, p-value of 0.019, and an R² value of 0.1076 that validates the integration of anatomical measurements into correlation analysis protocols. The scatter plot displays data points representing individual patient measurements with a linear regression line demonstrating the mathematical relationship between prostatic urethral angle measurements and pain ratings. The normal prostatic urethral angle of 35 degrees provides baseline reference measurements for correlation analysis, with deviations affecting R.I.D. Score adjustments through enhanced calculation methods. The correlation data supports the integration of anatomical measurements into R.I.D. Score calculations that determine treatment delivery parameters and influence overall correlation analysis between calculated injection factors and treatment outcomes.

Referring to FIG. 17, the procedure discomfort chart 980 presents comprehensive statistical analysis of the relationship between NPRS-11 scores and prostatic urethral angle measurements that contribute to R.I.D. Score correlation analysis. The procedure discomfort chart 980 includes coefficients and statistical data with standardized and unstandardized coefficients, standard error values, and confidence intervals that establish quantitative relationships between anatomical measurements and treatment outcome predictions. The statistical framework supports R.I.D. Score correlation analysis by providing validated measurement protocols that account for individual patient anatomical variations and procedural comfort factors. The data collection methodology eliminates the need to measure 1 cm increments from the bladder neck during procedures and reduces the need to enter the bladder during WVTT procedures through calculated positioning based on correlation analysis results. The comprehensive statistical analysis demonstrates that treatment planning protocols incorporating R.I.D. Score correlation data reduce the risk of acute urinary retention from 11-32% observed with standard protocols to 0% through precise correlation-based treatment parameter determination.

With continued reference to FIG. 2, the anatomical framework supporting R.I.D. Score correlation analysis involves the prostate tissue 230 surrounding the urethra 240, with the delivery device 260 providing controlled steam 220 distribution according to calculated correlation parameters. The prostate tissue 230 receives therapeutic intervention through controlled delivery of steam 220 heated to approximately 103 degrees Celsius, with each WVTT injection delivering approximately 0.5 mL of water vapor over 9 seconds according to correlation-based calculations. The delivery device 260 positioning within the urethra 240 enables precise steam 220 delivery according to R.I.D. Score correlation analysis that accounts for prostate volume measurements obtained through Computed Tomography Scan, Magnetic Resonance Imaging, or Ultrasound techniques. The spatial relationship between the bladder 210, the prostate 250, and the urethra 240 establishes anatomical constraints that influence correlation analysis between calculated injection parameters and treatment effectiveness measurements. The system includes an imaging device configured to obtain prostate volume measurements and a processor configured to calculate R.I.D. Score values based on correlation analysis data, with the imaging device including at least one of a Computed Tomography scanner, a Magnetic Resonance Imaging device, and an Ultrasound device for comprehensive correlation assessment protocols.

As illustrated in FIG. 3, the prostatic urethra 310 extends through the anatomical pathway that influences R.I.D. Score correlation analysis through geometric measurements and spatial relationships. The urethral angle 360 between the prostatic urethra 310 and the distal urethra 330 influences correlation calculations between anatomical measurements and treatment outcome predictions. The initial pressure point 320 and the secondary pressure point 340 establish measurement locations that contribute to correlation analysis between anatomical parameters and R.I.D. Score effectiveness measurements. The urethral diameter 350 establishes dimensional constraints that influence correlation analysis between delivery device positioning and treatment outcome assessments. The R.I.D. Score adjustments account for prostate anatomical variations through correlation analysis that incorporates the urethral angle 360 and other geometric parameters affecting treatment delivery effectiveness, with the R.I.D.+Score incorporating additional factors related to prostate anatomical variations beyond basic volume measurements for enhanced correlation analysis protocols.

Referring to FIG. 4, the prostate 250 demonstrates the urethral angle 360 measurement within the context of R.I.D. Score correlation analysis and treatment outcome assessment protocols. The urethral angle 360 measurement integrates with volume-based correlation calculations to establish enhanced treatment parameter determination that extends beyond basic dimensional assessments. The processor calculations account for the urethral angle 360 measurements through systematic correlation analysis that determines optimal treatment parameters based on individual patient anatomical configurations. The non-transitory computer-readable medium stores instructions that, when executed by a processor, cause the processor to perform correlation analysis incorporating anatomical measurements for determining treatment delivery parameters based on R.I.D. Score calculations. The correlation analysis demonstrates that prostatic urethral angle measurements function as predictive parameters for treatment effectiveness, enabling healthcare providers to implement calculated treatment protocols based on individual patient anatomical assessments and correlation-derived treatment parameters.

With reference to FIG. 5A, the first injection site 510, the second injection site 520, the third injection site 530, and the fourth injection site 540 establish spatial arrangements that correlate with R.I.D. Score calculations and treatment effectiveness measurements. The prostatic angle 550 influences correlation analysis between injection site positioning and treatment outcome assessments across the patient population. The injection site configuration correlates with R.I.D. Score factors ranging from 0.0025 to 0.6 injections per cubic centimeter, with preferred ranges of 0.025 to 0.1 injections per cubic centimeter and more preferred ranges of 0.045 to 0.055 injections per cubic centimeter based on correlation analysis results. FIGS. 5B-5D demonstrate magnetic resonance imaging visualization that supports correlation analysis between calculated injection site positioning and actual treatment delivery effectiveness. The imaging data validates the correlation between R.I.D. Score calculations and spatial distribution patterns within the prostate tissue 230, demonstrating the effectiveness of correlation-based treatment parameter determination across diverse patient anatomical configurations with prostate volumes ranging from 18 cc to 93 cc based on clinical correlation analysis data.

Referring to FIG. 10, the longitudinal treatment response demonstrates comprehensive symptom score improvements over time following WVTT procedures using R.I.D. Score calculations with specific treatment parameters. The graph displays three data points along a timeline including baseline, 4 weeks, and 4 months measurements that establish temporal relationships between R.I.D. Score-based treatment delivery and progressive symptom reduction patterns. The baseline measurement shows a symptom score of approximately 22, which decreases to approximately 7 at 4 weeks, and further decreases to approximately 4 at 4 months post-treatment. The graph includes error bars for each measurement point and indicates P-values of less than 0.0001 for both the 4-week and 4-month measurements, establishing statistical significance for treatment effectiveness across extended time intervals. The temporal progression demonstrates the sustained effectiveness of R.I.D. Score calculations in achieving progressive symptom improvement across extended treatment periods, with the R.I.D. Score factor of 0.05 injections per cubic centimeter providing consistent therapeutic outcomes.

With reference to FIG. 11, the baseline symptom score measurement establishes the initial clinical parameters for longitudinal analysis across the patient population undergoing water vapor thermal therapy. The graph displays a baseline measurement of 22.56±3.70 with a blue vertical bar representing the baseline score and an orange arrow pointing to the baseline value. The y-axis shows AUA symptom score values while the x-axis indicates the baseline measurement point, with error bars indicating the standard deviation from the baseline measurement. The baseline data provides the foundation for calculating symptom improvement correlations with R.I.D. Score factors ranging from 0.0025 to 0.6 injections per cubic centimeter. The standardized baseline measurement enables consistent longitudinal analysis between R.I.D.

Score calculations and treatment outcome assessments across diverse patient anatomical configurations with prostate volumes ranging from 18 cc to 93 cc based on clinical study data. The baseline symptom scores correlate with prostate volume measurements obtained through Computed Tomography Scan, Magnetic Resonance Imaging, or Ultrasound techniques that support R.I.D. Score calculations.

As shown in FIG. 12, the statistical analysis demonstrates symptom score improvements at 4 weeks post-treatment using R.I.D. Score calculations with a factor of 0.05 injections per cubic centimeter during longitudinal assessment protocols. The graph displays data points showing symptom score measurements over time with error bars indicating standard deviation values that validate the statistical significance of treatment outcomes during the initial follow-up period. The graph includes baseline measurements and subsequent measurements showing a decrease in symptom scores, with numerical values indicating a difference of 15.50±4.62 and a p-value of less than 0.0001. The measurement at the 4-week mark shows values of 7.06±3.07, demonstrating substantial symptom improvement following R.I.D. Score-based treatment delivery with water vapor heated to approximately 103 degrees Celsius. The statistical validation supports the effectiveness of the tested R.I.D. Score factor value of 0.05 injections per cubic centimeter in achieving clinically significant symptom improvements across the patient population during the initial longitudinal assessment period. Each WVTT injection delivers approximately 0.5 mL of water vapor over 9 seconds according to the calculated treatment parameters that correlate with symptom improvement measurements.

Referring to FIG. 13, the extended longitudinal analysis shows symptom score improvements at 4 months post-treatment using R.I.D. Score calculations with the tested factor of 0.05 injections per cubic centimeter. The graph illustrates changes in AUA symptom scores over time with data points showing a significant decrease in symptom scores from baseline measurements during extended follow-up assessment. The vertical axis represents AUA symptom score values while the horizontal axis represents time intervals, with the graph demonstrating a clear downward trend in symptom scores following R.I.D. Score-based treatment delivery. The statistical significance indicators show a p-value of less than 0.0001 with data showing a difference of 18.46±4.04 and a value of 3.91±2.36 at the 4-month time point. The longitudinal analysis validates the sustained effectiveness of R.I.D. Score calculations in maintaining symptom improvement across extended follow-up periods, demonstrating the long-term benefits of calculated injection parameters. The R.I.D. Score adjustments account for prostate anatomical variations through enhanced calculation methods that incorporate individual patient characteristics and correlate with sustained symptom improvement measurements throughout the longitudinal assessment period.

With continued reference to FIG. 8, the statistical analysis demonstrates quantitative relationships between R.I.D. Score values and symptom improvement measurements at 4 weeks post-treatment through scatter plot visualization and linear regression analysis during longitudinal assessment protocols. The graph displays data points representing the relationship between injections per milliliter of prostate volume on the x-axis and AUA SS change values on the y-axis derived from longitudinal clinical assessment protocols. The linear regression line shows the equation y=36.078x+13.751 with an R² value of 0.022, establishing mathematical relationships between R.I.D. Score factors and treatment outcomes across the patient population during the initial longitudinal follow-up period. The x-axis ranges from 0.0000 to 0.1200 injections/mL of prostate volume, encompassing R.I.D. Score factor ranges from 0.0025 to 0.6 injections per cubic centimeter with preferred ranges of 0.025 to 0.1 injections per cubic centimeter. The statistical correlation validates the effectiveness of R.I.D. Score factor calculations in predicting treatment outcomes during longitudinal assessment, with the tested value of 0.05 injections per cubic centimeter demonstrating consistent effectiveness in symptom improvement measurements across the clinical cohort. The longitudinal data collection eliminates the need to measure 1 cm increments from the bladder neck during procedures and reduces the need to enter the bladder during WVTT procedures through calculated positioning based on imaging-derived measurements.

As illustrated in FIG. 9, the graph 900 presents extended statistical analysis showing R.I.D. Score versus AUA SS change at 4 months post-treatment, demonstrating sustained treatment effectiveness over extended time periods during comprehensive longitudinal assessment. The graph 900 displays scatter plot data with a linear regression line where the x-axis represents injections/mL of prostate volume and the y-axis represents AUA SS change values measured through standardized longitudinal assessment protocols. The linear equation y=13.926x+17.844 with an R² value of 0.0045 establishes long-term correlation patterns between R.I.D. Score calculations and sustained symptom improvement across the patient cohort during extended longitudinal follow-up. The statistical analysis supports the R.I.D. Score factor calculation method involving dividing the average number of WVTT injections by the average prostate volume to establish standardized treatment parameters for longitudinal assessment protocols. The graph 900 validates the integration of R.I.D. Score calculations with treatment outcome measurements across extended follow-up periods, demonstrating the sustained effectiveness of calculated injection parameters throughout the longitudinal assessment timeline. The more preferred R.I.D. Score factor range of 0.045 to 0.055 injections per cubic centimeter correlates with sustained symptom improvement measurements during extended longitudinal analysis periods.

Referring to FIG. 7A, the symptom scoresheet 700 establishes the standardized measurement framework for quantifying treatment outcomes during longitudinal R.I.D. Score assessment protocols. The symptom scoresheet 700 contains multiple questions related to urinary function with corresponding numerical rating scales ranging from 0 to 5, where patients indicate symptom frequency over specified time periods for longitudinal assessment evaluation. The scoresheet evaluation encompasses sections for incomplete emptying, frequency, intermittency, urgency, weak stream, straining, and nocturia symptoms that correlate directly with R.I.D. Score calculations and treatment effectiveness measurements throughout longitudinal follow-up periods. The numerical rating system within the symptom scoresheet 700 provides quantitative data for statistical analysis and longitudinal assessment across patient populations with diverse prostate anatomical configurations. The longitudinal assessment protocols demonstrate that the method reduces the risk of acute urinary retention from 11-32% to 0% compared to standard protocols through precise R.I.D. Score-based treatment parameter determination. The symptom scoresheet 700 measurements correlate with R.I.D.+Score calculations that incorporate additional factors related to prostate anatomical variations beyond basic volume measurements for enhanced longitudinal assessment protocols.

With reference to FIG. 6, the clinical validation process establishes the methodological framework for longitudinal R.I.D. Score assessment through systematic patient selection and data collection protocols across extended time periods. The first step 610 encompasses single-surgeon experience with procedures conducted between October 2023 and June 2024, establishing standardized treatment protocols across a defined temporal period for longitudinal analysis evaluation. The cohort characteristics include 60 patients with mean age of 67.4±6.62 years, prostate volume of 54.86±25.40 cc, PSA levels of 2.38±2.38 ng/mL, and prostatic urethral angle measurements of 39.43±10.41 degrees that support longitudinal assessment protocols. The second step 620 details patient exclusion criteria affecting longitudinal analysis integrity, with four patients excluded based on non-standard pain protocol implementation and chronic Foley catheter usage patterns that could influence extended follow-up measurements. The third step 630 involves comprehensive measurement protocols using 7.5 MHz ultrasound with a transrectal probe for accurate prostate volume determination that supports longitudinal R.I.D. Score assessment calculations. Linear regression models and paired T-tests were conducted using SPSS software to establish statistical relationships between prostate volume measurements and treatment outcomes during longitudinal assessment periods, incorporating AUA SS and NPRS-11 metrics for comprehensive longitudinal analysis evaluation.

As shown in FIG. 2, the anatomical framework supporting longitudinal treatment response involves the prostate tissue 230 surrounding the urethra 240, with the delivery device 260 providing controlled steam 220 distribution according to calculated longitudinal assessment parameters. The prostate tissue 230 receives therapeutic intervention through controlled delivery of steam 220 heated to approximately 103 degrees Celsius, with each WVTT injection delivering approximately 0.5 mL of water vapor over 9 seconds according to longitudinal assessment-based calculations. The delivery device 260 positioning within the urethra 240 enables precise steam 220 delivery according to R.I.D. Score longitudinal assessment analysis that accounts for prostate volume measurements obtained through Computed Tomography Scan, Magnetic Resonance Imaging, or Ultrasound techniques. The spatial relationship between the bladder 210, the prostate 250, and the urethra 240 establishes anatomical constraints that influence longitudinal assessment between calculated injection parameters and treatment effectiveness measurements across extended time periods. The system includes an imaging device configured to obtain prostate volume measurements and a processor configured to calculate R.I.D. Score values based on longitudinal assessment data, with the imaging device including at least one of a Computed Tomography scanner, a Magnetic Resonance Imaging device, and an Ultrasound device for comprehensive longitudinal assessment protocols. The non-transitory computer-readable medium stores instructions that, when executed by a processor, cause the processor to perform longitudinal assessment calculations incorporating anatomical measurements for determining treatment delivery parameters based on R.I.D. Score calculations across extended follow-up periods.

Referring to FIG. 3, the prostatic urethra 310 extends through the anatomical pathway that influences longitudinal R.I.D. Score assessment through geometric measurements and spatial relationships during extended treatment evaluation periods. The urethral angle 360 between the prostatic urethra 310 and the distal urethra 330 influences longitudinal assessment calculations between anatomical measurements and treatment outcome predictions across extended time intervals. The initial pressure point 320 and the secondary pressure point 340 establish measurement locations that contribute to longitudinal assessment analysis between anatomical parameters and R.I.D. Score effectiveness measurements throughout follow-up periods. The urethral diameter 350 establishes dimensional constraints that influence longitudinal assessment analysis between delivery device positioning and treatment outcome assessments during extended evaluation periods. The normal prostatic urethral angle of 35 degrees provides baseline reference measurements for longitudinal assessment protocols, with deviations affecting R.I.D. Score adjustments through enhanced calculation methods during extended follow-up evaluation. The prostatic urethral angle measurement functions as a predictive tool for procedure discomfort levels and correlates with longitudinal treatment response patterns across extended assessment periods.

With continued reference to FIG. 5A, the first injection site 510, the second injection site 520, the third injection site 530, and the fourth injection site 540 establish spatial arrangements that correlate with longitudinal R.I.D. Score assessment and treatment effectiveness measurements across extended time periods. The prostatic angle 550 influences longitudinal assessment analysis between injection site positioning and treatment outcome assessments across the patient population during extended follow-up evaluation. The injection site configuration correlates with R.I.D. Score factors ranging from 0.0025 to 0.6 injections per cubic centimeter, with preferred ranges of 0.025 to 0.1 injections per cubic centimeter and more preferred ranges of 0.045 to 0.055 injections per cubic centimeter based on longitudinal assessment analysis results. FIGS. 5B-5D demonstrate magnetic resonance imaging visualization that supports longitudinal assessment analysis between calculated injection site positioning and actual treatment delivery effectiveness across extended evaluation periods. The imaging data validates the correlation between R.I.D. Score calculations and spatial distribution patterns within the prostate tissue 230, demonstrating the effectiveness of longitudinal assessment-based treatment parameter determination across diverse patient anatomical configurations with prostate volumes ranging from 18 cc to 93 cc based on clinical longitudinal assessment analysis data throughout extended follow-up periods.

Referring to FIG. 14, the pain score rating chart demonstrates the practical application of numerical assessment tools in clinical settings for water vapor thermal therapy procedures. The chart displays a numerical scale from 0 to 10 with a circular indicator positioned at approximately level 3, illustrating the measurement methodology used during patient assessment protocols. The scale incorporates a gradient coloring scheme that transitions from light gray at the low end to black at the high end of the pain scale, providing visual reference points that correspond to increasing discomfort levels. The pain score rating chart functions in conjunction with the symptom scoresheet 700 to provide comprehensive patient assessment data that supports R.I.D. Score calculations and treatment planning protocols. The measurement system accounts for individual patient anatomical variations through the prostatic urethral angle measurement system, which serves to predict procedure discomfort levels and guide appropriate comfort management protocols. The prostatic urethral angle measurement can be used to predict procedure discomfort levels during delivery device insertion and steam 220 delivery procedures, with the normal prostatic urethral angle of 35 degrees providing baseline reference measurements for discomfort prediction calculations.

With reference to FIG. 15, the enhanced pain score rating chart presents a categorized assessment system that divides patient discomfort into three distinct ranges: mild discomfort ranging from 0-3, moderate discomfort ranging from 4-6, and pain ranging from 7-10. The chart includes color-coded indicators with yellow, orange, and red circles containing numerical values of 38, 13, and 1 respectively, positioned along the scale to represent patient distribution patterns across discomfort categories. The categorized system provides healthcare providers with standardized reference points for implementing appropriate procedural modifications based on predicted discomfort levels derived from prostatic urethral angle measurements. The pain assessment categories correlate with R.I.D. Score adjustments that account for prostate anatomical variations through calculations incorporating the prostatic urethral angle and other geometric parameters affecting treatment delivery. The measurement system supports the method that reduces the risk of acute urinary retention from 11-32% to 0% compared to standard protocols through precise anatomical assessment and comfort prediction protocols. The R.I.D. Score factor ranges from 0.0025 to 0.6 injections per cubic centimeter, with preferred ranges of 0.025 to 0.1 injections per cubic centimeter and more preferred ranges of 0.045 to 0.055 injections per cubic centimeter that correlate with discomfort prediction accuracy.

As shown in FIG. 16, the correlation between prostatic urethral angles and numeric pain rating scale scores establishes quantitative relationships between anatomical measurements and procedural discomfort across clinical patient populations. The statistical analysis indicates a positive correlation described by the linear equation y=0.0436x+1.3003, with a standard coefficient of 0.328, p-value of 0.019, and an R² value of 0.1076 that validates the predictive capabilities of angular measurements in treatment planning. The scatter plot displays data points representing individual patient measurements with a linear regression line demonstrating the mathematical relationship between prostatic urethral angle measurements in degrees and pain ratings on the numeric scale from 0-8. The correlation data supports the integration of angular measurements into R.I.D. Score calculations that determine treatment delivery parameters and patient comfort predictions based on individual anatomical configurations. The statistical validation demonstrates that treatment planning protocols incorporating prostatic urethral angle measurements enable healthcare providers to implement appropriate comfort management strategies based on predicted discomfort levels. The tested R.I.D. Score factor value of 0.05 injections per cubic centimeter correlates with specific discomfort prediction patterns across the patient population, with water vapor delivered at approximately 103 degrees Celsius during WVTT procedures according to calculated comfort parameters.

Referring to FIG. 17, the procedure discomfort chart 980 presents comprehensive statistical analysis of the relationship between NPRS-11 scores and prostatic urethral angle measurements across clinical validation cohorts. The procedure discomfort chart 980 includes coefficients and statistical data with standardized and unstandardized coefficients, standard error values, and confidence intervals that establish quantitative relationships between anatomical measurements and patient comfort predictions during water vapor thermal therapy procedures. The statistical analysis validates the effectiveness of ultrasound imaging performed using a transrectal probe at 7.5 MHz frequency for accurate anatomical assessment and discomfort prediction protocols that support R.I.D. Score calculations. The data collection methodology within the procedure discomfort chart 980 eliminates the need to measure 1 cm increments from the bladder neck during procedures and reduces the need to enter the bladder 210 during WVTT procedures through calculated positioning based on imaging-derived measurements. The comprehensive statistical framework supports R.I.D. Score factor calculations ranging from 0.0025 to 0.6 injections per cubic centimeter, with preferred ranges of 0.025 to 0.1 injections per cubic centimeter and more preferred ranges of 0.045 to 0.055 injections per cubic centimeter. Each WVTT injection delivers approximately 0.5 mL of water vapor over 9 seconds according to discomfort-adjusted treatment parameters derived from the procedure discomfort chart 980 analysis.

With continued reference to FIG. 3, the prostatic urethra 310 extends through the anatomical pathway that influences discomfort prediction calculations through geometric measurements and spatial relationships during water vapor thermal therapy procedures. The urethral angle 360 between the prostatic urethra 310 and the distal urethra 330 influences discomfort prediction calculations between anatomical measurements and patient comfort assessments during treatment delivery. The initial pressure point 320 and the secondary pressure point 340 establish measurement locations that contribute to discomfort prediction analysis between anatomical parameters and procedural comfort assessments. The urethral diameter 350 establishes dimensional constraints that influence discomfort prediction analysis between delivery device 260 positioning and patient comfort assessments during treatment procedures. The normal prostatic urethral angle of 35 degrees provides baseline reference measurements for discomfort prediction protocols, with deviations affecting R.I.D. Score adjustments through enhanced calculation methods that incorporate comfort prediction parameters. The prostatic urethral angle measurement functions as a predictive tool for procedure discomfort levels and correlates with patient comfort patterns across different anatomical configurations, with larger angles typically associated with increased procedural discomfort during delivery device 260 insertion and steam 220 delivery procedures.

As illustrated in FIG. 7B, the pain assessment scale establishes a numerical rating system ranging from 0 to 10 with specific gradations that correspond to increasing levels of patient discomfort during water vapor thermal therapy procedures. The scale presents a horizontal gradient that transitions from “No pain” at the 0 mark through “Moderate pain” at the midpoint to “Worst possible pain” at the 10 mark, providing clear reference points for patient comfort assessment during treatment delivery. The numerical pain rating scale correlates directly with prostatic urethral angle measurements for predicting procedure discomfort levels during delivery device 260 insertion and steam 220 delivery procedures within the prostate tissue 230. The normal prostatic urethral angle of 35 degrees provides baseline reference measurements, with deviations from this standard measurement affecting discomfort predictions and requiring R.I.D. Score adjustments through enhanced calculation methods that incorporate comfort prediction parameters. The pain assessment scale integrates with the prostatic urethral angle measurement system to predict procedure discomfort levels, with larger angles typically associated with increased patient discomfort during therapeutic intervention through the urethra 240. The R.I.D. Score adjustments account for prostate anatomical variations through calculations that incorporate the prostatic urethral angle and other geometric parameters affecting treatment delivery comfort, with the R.I.D.+Score incorporating additional factors related to prostate anatomical variations beyond basic volume measurements for enhanced discomfort prediction protocols.

Referring to FIG. 6, the third step 630 involves anatomical measurement protocols using 7.5 MHz ultrasound with a transrectal probe for accurate prostatic urethral angle assessment and discomfort prediction analysis during clinical validation procedures. The ultrasound imaging protocols provide precise measurements of prostatic urethral angles and dimensional parameters that support discomfort prediction calculations and treatment planning procedures across the patient population. The imaging device includes at least one of a Computed Tomography scanner, a Magnetic Resonance Imaging device, and an Ultrasound device for obtaining comprehensive anatomical measurements that guide angular analysis and discomfort prediction calculations. The measurement protocols established in the third step 630 provide standardized approaches for angular assessment and discomfort prediction across different patient populations and clinical settings, ensuring consistent data collection for R.I.D. Score calculation systems that incorporate comfort prediction parameters. The clinical validation process demonstrates that the method reduces the risk of acute urinary retention from 11-32% to 0% compared to standard protocols through precise discomfort prediction and anatomical assessment procedures. The R.I.D. Score factor calculation involves dividing the average number of WVTT injections by the average prostate volume, establishing standardized relationships between anatomical measurements and discomfort prediction parameters across patient populations with prostate volumes ranging from 18 cc to 93 cc based on clinical study data.

With reference to FIG. 4, the prostate 250 demonstrates the urethral angle 360 measurement within the context of discomfort prediction analysis and comprehensive anatomical assessment protocols during treatment planning procedures. The urethral angle 360 measurement integrates with volume-based discomfort prediction calculations to establish enhanced treatment parameter determination that extends beyond basic dimensional assessments and incorporates comfort prediction protocols. The processor calculations account for the urethral angle 360 measurements through systematic discomfort prediction analysis that determines optimal treatment parameters based on individual patient anatomical configurations and comfort assessment requirements. The non-transitory computer-readable medium stores instructions that, when executed by a processor, cause the processor to perform discomfort prediction analysis incorporating anatomical measurements for determining treatment delivery parameters based on R.I.D. Score calculations and comfort assessment protocols. The discomfort prediction analysis demonstrates that prostatic urethral angle measurements function as reliable predictors for procedural comfort levels, enabling healthcare providers to implement calculated treatment protocols based on individual patient anatomical assessments and discomfort prediction parameters. The system includes an imaging device configured to obtain prostate volume measurements and a processor configured to calculate R.I.D. Score values based on discomfort prediction analysis data, with the imaging device including at least one of a Computed Tomography scanner, a Magnetic Resonance Imaging device, and an Ultrasound device for comprehensive discomfort prediction assessment protocols.

As shown in FIG. 2, the anatomical framework supporting discomfort prediction analysis involves the prostate tissue 230 surrounding the urethra 240, with the delivery device 260 providing controlled steam 220 distribution according to calculated discomfort prediction parameters during treatment procedures. The prostate tissue 230 receives therapeutic intervention through controlled delivery of steam 220 heated to approximately 103 degrees Celsius, with each WVTT injection delivering approximately 0.5 mL of water vapor over 9 seconds according to discomfort prediction-based calculations that account for patient comfort assessments. The delivery device 260 positioning within the urethra 240 enables precise steam 220 delivery according to discomfort prediction analysis that accounts for prostate volume measurements obtained through Computed Tomography Scan, Magnetic Resonance Imaging, or Ultrasound techniques. The spatial relationship between the bladder 210, the prostate 250, and the urethra 240 establishes anatomical constraints that influence discomfort prediction analysis between calculated injection parameters and patient comfort assessments during treatment delivery. The delivery device 260 operation reduces the need to enter the bladder 210 during WVTT procedures through calculated positioning based on discomfort prediction analysis that incorporates anatomical measurements and comfort assessment protocols. The steam 220 distribution patterns within the prostate tissue 230 correlate with discomfort prediction calculations that account for the prostatic urethral angle and other geometric parameters affecting patient comfort during therapeutic intervention through the delivery device 260 positioning within the urethra 240.

A number of implementations have been described. Nevertheless, it will be understood that various modifications may be made without departing from the spirit and scope of the disclosure. Accordingly, other implementations are within the scope of the following claims.

The system and method can further be described as a method for determining a number of water vapor thermal therapy (WVTT) injections for treatment of benign prostate hyperplasia, comprising:
a. obtaining a prostate volume of a patient; and
b. calculating a R.I.D. Score by multiplying the prostate volume by a R.I.D. Score factor, wherein the R.I.D. Score indicates the number of WVTT injections required for treatment.

The method wherein the R.I.D. Score factor is within a range of 0.0025 to 0.6 injections per cubic centimeter.

The method wherein the R.I.D. Score factor is within a range of 0.025 to 0.1 injections per cubic centimeter.

The method wherein the R.I.D. Score factor is within a range of 0.045 to 0.055 injections per cubic centimeter.

The method wherein obtaining the prostate volume comprises using at least one imaging technique selected from the group consisting of Computed Tomography Scan, Magnetic Resonance Imaging, and Ultrasound.

The method wherein the Ultrasound uses a transrectal probe at 7.5 MHz frequency.

The method further comprises a step of adjusting the R.I.D. Score to account for prostate anatomical variations of the patient.

The method wherein adjusting the R.I.D. Score comprises calculating a R.I.D.+Score that incorporates additional factors related to the prostate anatomical variations.

The method wherein the prostate anatomical variations comprise prostatic urethral angle measurements.

The method wherein the R.I.D. Score factor is calculated by dividing an average number of WVTT injections by an average prostate volume.

A system for determining a number of water vapor thermal therapy (WVTT) injections for treatment of benign prostate hyperplasia, comprising:
a. an imaging device configured to obtain a prostate volume of a patient; and
b. a processor configured to calculate a R.I.D. Score by multiplying the prostate volume by a R.I.D. Score factor, wherein the R.I.D. Score indicates the number of WVTT injections required for treatment.

The system wherein the R.I.D. Score factor is within a range of 0.0025 to 0.6 injections per cubic centimeter.

The system wherein the R.I.D. Score factor is within a range of 0.025 to 0.1 injections per cubic centimeter.

The system wherein the imaging device comprises at least one of a Computed Tomography scanner, a Magnetic Resonance Imaging device, and an Ultrasound device.

The system wherein the Ultrasound device uses a transrectal probe at 7.5 MHz frequency.

The system wherein the processor is further configured to adjust the R.I.D. Score to account for prostate anatomical variations of the patient.

A non-transitory computer-readable medium storing instructions that, when executed by a processor, cause the processor to perform a method for determining a number of water vapor thermal therapy (WVTT) injections for treatment of benign prostate hyperplasia, the method comprising:
a. receiving a prostate volume of a patient; and
b. calculating a R.I.D. Score by multiplying the prostate volume by a R.I.D. Score factor, wherein the R.I.D. Score indicates the number of WVTT injections required for treatment.

The non-transitory computer-readable medium, wherein the R.I.D. Score factor is within a range of 0.0025 to 0.6 injections per cubic centimeter.

The non-transitory computer-readable medium wherein the R.I.D. Score factor is within a range of 0.045 to 0.055 injections per cubic centimeter.

The non-transitory computer-readable medium wherein the method further comprises a step of adjusting the R.I.D. Score to account for prostate anatomical variations of the patient.

Referring now to the drawings FIG. 1-7, and more particularly to FIG. 1, there is shown the graph of AUH BPH Guidelines process which starts with Surgical Therapy step 110 and the step 120 where an assessment of prostate size via imaging or cystoscopy. If it is a large prostate(>80-150 cc) or very large prostate(>150 cc) the healthcare professional uses step 130 to evaluate the prostate using Simple Prostatectomy (open Laparoscopic, robotic), Holmium Laser Enucleation of the Prostate (HoLEP) which is a minimally invasive surgery for benign prostatic hyperplasia (BPH), an enlarged prostate that obstructs urine flow or Thulium Laser Enucleation of the Prostate (ThyLEP) which is a minimally invasive surgery for treating benign prostatic hyperplasia (BPH) (an enlarged prostate). Based on the results of the evaluation the healthcare professional uses step 140 to implement WVTT step 140.

If the Prostate is average in size (30-80 CC) then healthcare professional uses the Step 140 to operate on the prostate using robotic water jet ablation (RWT), HoLEP, Photoselective Vaporization of the Prostate (PVP), Prostatic Urethral Lift (PUL), Thulium Laser Enucleation of the Prostate, (ThuLEP), Temporary Implanted Prostatic Devices (TIPD), Transurethral Resection of the Prostate (TURP), Transurethral vaporization of the prostate (TUVP) and Water Vapor Thermal Therapy (WVTT).

If the Prostate is small in size (<30 CC) then healthcare professional uses Step 150 to operate on the prostate using HoLEP, PVP, ThuLEP, TURP, TUVP, TIPD or Transurethral incision of the prostate (TUIP).

FIG. 2 shows a cross section of a prostate and anatomy parts surrounding the prostate. Bladder 210, Steam 220, Prostate tissue 230, Urethra 240, Prostate 250 and delivery device 260.

WVTT Standard procedure includes Rigid Cystoscope 12 Fr with 30° lens, water vapor at about 103° C., 9-second injections (each 0.5 mL), Treatment every 1 cm, and total injections by length of the prostatic urethra. The average number of injections using the procedure is 4.7 injection plus or minus 1.7 injections.

One problem with standard treatment protocol recently used was Acute Urinary Retention. AUR is about 11-32%. In one study of 18 patients, mean prostate volume 45.98 cc with a volume range of 18-93 cc. There were 4 patients representing 22% of the total number of patients who failed trial without catheter (TWOC) after procedure. Three patients were not able to void. One patient had PVR>300 mL. Another problem with standard treatment protocol recently used was procedure discomfort. The office-based procedure included discomfort of procedure upon insertion of cystoscope, on angulation in prostatic urethra and on each treatment injection. As shown in the diagram of FIG. 3 and scan of FIG. 4, two factors improve comfort, the prostatic urethral angle (PUA) and the number of WVTT injections. PUA as an influencing factor in lower urinary tract symptoms. The normal prostatic urethral angle is 35 degrees. Discomfort during in-office Rigid Cystoscopy is well known using the urethral angle of 35 degrees. FIG. 3 FIG. 4 shows the bladder 250, P1 (initial pressure) and V1 (initial volume) 320, Prostatic Urethra 310, Distal Urethra 330, P2,V2 340, diameter of the urethra 350, Prostatic Urethral Angle 360.

FIGS. 5A, 5B, 5C and 5D show scans of the injection site 510, 520, 530 and 540. Average length of Prostatic Urethra: 4.5-5.0 cm. This would have previously prompted a recommendation of 4-5 injections for the procedure. The R.I.D. score formula allows for a more accurate calculation of the recommended number of injections in order to minimize discomfort in a patient.

The R.I.D. score formula predicts number of injections needed based on prostate volume prior to the procedure. There is no need to go into bladder during the procedure. There is no need to measure 1 cm increments from bladder neck. There is a reduced risk of overtreatment, reduced risk of irritative symptoms, reduced risk of fever and chills and a reduced risk of problematic gross hematuria.

FIG. 6 is a chart of a cohort study with ultrasound with a transrectal probe at 7.5 MHz. Cohort characteristics include 60 patients (mean±SD) Age: 67.4±6.62 years, prostate volume: 54.86±25.40 cc, PSA: 2.38±2.38 ng/mL and PUA: 39.43±10.41 degrees. Step 610 includes Single-surgeon experience and Underwent procedure between October 2023 and June 2024, Step 620 includes 4 Patients Excluded, the Criteria (Non-standard Pain Protocol and Chronic Foley User), step 620 includes 7.5 MHz Ultrasound with Transrectal Probe, Linear Regression Models and Paired T-Tests were conducted using SPSS, AUA SS and NPRS-11

FIG. 7 A is a graph 700 showing AUA symptom score (SS) and measure of pain intensity using the NPRS-11 scale FIG. 7 B.

FIG. 8 is a graph 800 showing RID Score vs AUA symptom score changes at 4 weeks.

FIG. 9 is a Graph 900 Showing Rid Score Vs Aua Symptom Score changes at 4 months.

FIG. 10 is a graph 910 showing AUA symptom score vs. time after TWVT with RID score if 0.05.

FIG. 11 is a graph 920 showing AUA symptom score with Baseline at 22.56 plus or minus 3.70.

FIG. 12 is a graph 930 showing AUA symptom score vs. time after TWVT with RID score if 0.05. changes at 4 weeks.

FIG. 13 is a graph 940 showing AUA symptom score vs. time after TWVT with RID score if 0.05. changes at 4 months.

FIG. 14 is a graph 950 showing pain score rating chart.

FIG. 15 is a graph 960 showing pain score rating chart.

FIG. 16 is a chart 970 showing prostatic Urethral Angles vs. numeric pain rating scale 11.

FIG. 17 is a chart 980 showing procedure discomfort values.

Adverse events shown in the studies of the above charts, Foley use after WVTT: 4.80±0.75 days. Post WVTT urinary retention: 0%. No Failure of TWOC. No patient with PVR>300 mL. Post WVTT febrile episodes: 0%. Post WVTT significant gross hematuria episodes: 0%.

Conclusion of the charts show WVTT using low RID Scores is an effective treatment for BPH with LUTS. Using Low RID Score of 0.05 there is clinically significant change in AUA SS at 1 and 4 months, improved rates of urinary retention of 11-32% to 0%, higher PUA relates to more pain (NPRS-11). Pre-op TRUS findings can decide pain management protocol.

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A method for determining a number of water vapor thermal therapy (WVTT) injections for treatment of benign prostate hyperplasia, comprising:
obtaining a prostate volume of a patient; and
calculating a R.I.D. Score by multiplying the prostate volume by a R.I.D. Score factor, wherein the R.I.D. Score indicates the number of WVTT injections required for treatment.

2. The method of claim 1, wherein the R.I.D. Score factor is within a range of 0.0025 to 0.6 injections per cubic centimeter.

3. The method of claim 2, wherein the R.I.D. Score factor is within a range of 0.025 to 0.1 injections per cubic centimeter.

4. The method of claim 3, wherein the R.I.D. Score factor is within a range of 0.045 to 0.055 injections per cubic centimeter.

5. The method of claim 1, wherein obtaining the prostate volume comprises using at least one imaging technique selected from the group consisting of Computed Tomography Scan, Magnetic Resonance Imaging, and Ultrasound.

6. The method of claim 5, wherein the Ultrasound uses a transrectal probe at 7.5 MHz frequency.

7. The method of claim 1, further comprising a step of adjusting the R.I.D. Score to account for prostate anatomical variations of the patient.

8. The method of claim 7, wherein adjusting the R.I.D. Score comprises calculating a R.I.D.+Score that incorporates additional factors related to the prostate anatomical variations.

9. The method of claim 8, wherein the prostate anatomical variations comprise prostatic urethral angle measurements.

10. The method of claim 1, wherein the R.I.D. Score factor is calculated by dividing an average number of WVTT injections by an average prostate volume.

11. A system for determining a number of water vapor thermal therapy (WVTT) injections for treatment of benign prostate hyperplasia, comprising:
an imaging device configured to obtain a prostate volume of a patient; and
a processor configured to calculate a R.I.D. Score by multiplying the prostate volume by a R.I.D. Score factor, wherein the R.I.D. Score indicates the number of WVTT injections required for treatment.

12. The system of claim 11, wherein the R.I.D. Score factor is within a range of 0.0025 to 0.6 injections per cubic centimeter.

13. The system of claim 12, wherein the R.I.D. Score factor is within a range of 0.025 to 0.1 injections per cubic centimeter.

14. The system of claim 11, wherein the imaging device comprises at least one of a Computed Tomography scanner, a Magnetic Resonance Imaging device, and an Ultrasound device.

15. The system of claim 14, wherein the Ultrasound device uses a transrectal probe at 7.5 MHz frequency.

16. The system of claim 11, wherein the processor is further configured to adjust the R.I.D. Score to account for prostate anatomical variations of the patient.

17. A non-transitory computer-readable medium storing instructions that, when executed by a processor, cause the processor to perform a method for determining a number of water vapor thermal therapy (WVTT) injections for treatment of benign prostate hyperplasia, the method comprising:
receiving a prostate volume of a patient; and
calculating a R.I.D. Score by multiplying the prostate volume by a R.I.D. Score factor, wherein the R.I.D. Score indicates the number of WVTT injections required for treatment.

18. The non-transitory computer-readable medium of claim 17, wherein the R.I.D. Score factor is within a range of 0.0025 to 0.6 injections per cubic centimeter.

19. The non-transitory computer-readable medium of claim 18, wherein the R.I.D. Score factor is within a range of 0.045 to 0.055 injections per cubic centimeter.

20. The non-transitory computer-readable medium of claim 17, wherein the method further comprises a step of adjusting the R.I.D. Score to account for prostate anatomical variations of the patient.
